SEQUENCE LISTING (38 sequences)

SEQ ID NO: 1 - protein, 736 aa, Adeno-associated virus serotype 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - protein, 724 aa, Adeno-associated virus serotype 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 3 - protein, 15 aa, synthetic construct
GFRPKRLNFKLFNIQ

SEQ ID NO: 4 - protein, 15 aa, synthetic construct
PKRLNFKLFNIQVKE

SEQ ID NO: 5 - protein, 15 aa, synthetic construct
LNFKLFNIQVKEVTD

SEQ ID NO: 6 - protein, 21 aa, synthetic construct
GFRPKRLNFKLFNIQVKEVTD

SEQ ID NO: 7 - protein, 21 aa, synthetic construct
GFRPKRLNFKLFNIQVKEVTT

SEQ ID NO: 8 - protein, 21 aa, synthetic construct
GFRPKRLNFKLFNIQVKEVTQ

SEQ ID NO: 9 - protein, 21 aa, synthetic construct
GFRPKKLSFKLFNIQVRGVTQ

SEQ ID NO: 10 - protein, 21 aa, synthetic construct
GMRPKAMRVKIFNIQVKEVTT

SEQ ID NO: 11 - protein, 21 aa, synthetic construct
GFRPRSLRVKIFNIQVKEVTV

SEQ ID NO: 12 - protein, 21 aa, synthetic construct
GFRPKRLNFKLFNIQVKEVTT

SEQ ID NO: 13 - protein, 21 aa, synthetic construct
GFRPKKLRFKLFNIQVKEVTT

SEQ ID NO: 14 - protein, 21 aa, synthetic construct
GFRPKRLSFKLFNIQVKEVTQ

SEQ ID NO: 15 - protein, 21 aa, synthetic construct
GFRPKRLSFKLFNIQVKEVTQ

SEQ ID NO: 16 - protein, 21 aa, synthetic construct
GLRPKAMRVKIFNIQVKEVTT

SEQ ID NO: 17 - protein, 20 aa, synthetic construct
GLRPKSMRVIFNIQVKEVTT

SEQ ID NO: 18 - protein, 19 aa, synthetic construct
GFRPRLNFKLFNIQVEVTQ

SEQ ID NO: 19 - protein, 15 aa, synthetic construct
KRLNFKLFNIQVKEV

SEQ ID NO: 20 - protein, 15 aa, synthetic construct
KRLRFKLFNIQVKEV

SEQ ID NO: 21 - protein, 15 aa, synthetic construct
KRLNVKLFNIQVKEV

SEQ ID NO: 22 - protein, 15 aa, synthetic construct
KRLNFKIFNIQVKEV

SEQ ID NO: 23 - protein, 15 aa, synthetic construct
KRLRVKLFNIQVKEV

SEQ ID NO: 24 - protein, 15 aa, synthetic construct
KRLRFKIFNIQVKEV

SEQ ID NO: 25 - protein, 15 aa, synthetic construct
KRLNVKIFNIQVKEV

SEQ ID NO: 26 - protein, 15 aa, synthetic construct
KRLRVKIFNIQVKEV

SEQ ID NO: 27 - protein, 15 aa, synthetic construct
RSLRVKIFNIQVKEV

SEQ ID NO: 28 - protein, 15 aa, synthetic construct
NNYWGFRPRSLRVKI

SEQ ID NO: 29 - protein, 15 aa, synthetic construct
WGFRPRSLRVKIFNI

SEQ ID NO: 30 - protein, 15 aa, synthetic construct
RPRSLRVKIFNIQVK

SEQ ID NO: 31 - protein, 15 aa, synthetic construct
SLRVKIFNIQVKEVT

SEQ ID NO: 32 - protein, 15 aa, synthetic construct
VKIFNIQVKEVTVQD

SEQ ID NO: 33 - protein, 15 aa, synthetic construct
NNWGFRPKRLNFKLF

SEQ ID NO: 34 - protein, 15 aa, synthetic construct
KLFNIQVKEVTDNNG

SEQ ID NO: 35 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNVKIFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 36 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPRSLRVKLINIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 37 - DNA, 2211 bp, synthetic construct
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacgtcaagatcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 38 - DNA, 2211 bp, synthetic construct
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctcggtccctcagagtcaagatcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

(19) United States
(12) Patent Application Publication
(10) Pub. No.: US 2026/0347877 A1
(43) Pub. Date: Nov. 5, 2026

(54) RECOMBINANT ADENO-ASSOCIATED VIRUS VECTORS LACKING AN IMMUNODOMINANT T CELL EPITOPE AND USE THEREOF

(71) Applicant: The U.S.A., as represented by the Secretary, Department of Health and Human Services, Silver Spring, MD (US)

(72) Inventors: Ronit Jolles-Mazor, Rockville, MD (US); Sojin Bing, Rockville, MD (US)

(73) Assignee: The U.S.A., as represented by the Secretary, Department of Health and Human Services, Silver Spring, MD (US)

(21) Appl. No.: 19/158,216

(22) PCT Filed: Feb. 21, 2024
(86) PCT No.: PCT/US2024/016716

§ 371 Date: Aug. 20, 2025

Related U.S. Application Data
(60) Provisional application No. 63/486,299, filed on Feb. 22, 2023.

(51) Int. Cl. C12N 15/86; A61K 48/00; C07K 14/005

(52) U.S. Cl. CPC C12N 15/86; A61K 48/0008; C07K 14/005; C12N 2750/14122; C12N 2750/14134; C12N 2750/14143

(57) ABSTRACT

Recombinant adeno-associated virus (AAV) vectors encoding a modified VP1 protein lacking an immunodominant T cell epitope, as well as AAV vector particles containing the modified VP1 protein, are described. Use of the recombinant AAV vectors and vector particles as improved gene therapy vectors with reduced immunogenicity is also described. Isolated VP1 peptides containing an immunodominant T cell epitope, and use thereof, is further described.

### CROSS REFERENCE TO RELATED APPLICATIONS

This application is the U.S. National Stage of International Application No. PCT/US2024/016716, filed Feb. 21, 2024, which was published in English under PCT Article 21(2), which claims the benefit of U.S. Provisional Application No. 63/486,299, filed Feb. 22, 2023. The above-referenced applications are herein incorporated by reference in their entirety.

### FIELD

This disclosure concerns recombinant adeno-associated virus (AAV) vectors expressing a modified form of capsid protein VP1 that lacks an immunodominant CD4 T cell epitope. This disclosure further concerns use of the recombinant AAV vectors, such as in gene therapy applications.

### INCORPORATION OF ELECTRONIC SEQUENCE LISTING

The electronic sequence listing, submitted herewith as an XML file named 9531-109705-03_revised (41,692 bytes), created on Jun. 16, 2026, is herein incorporated by reference in its entirety.

### BACKGROUND

Gene therapy using recombinant adeno-associated virus (AAV) vectors is one of the most promising approaches for treating a wide range of inherited and acquired diseases (Mingozzi and High, Nat Rev Genet 12:341-355, 2011). However, the safety and effectiveness of AAV vector-mediated gene transfer in humans are influenced by immune responses against AAV capsid proteins, with some cases leading to acute toxicities and loss of efficacy (Mingozzi et al., Nat Med 13:419-422, 2007; Verdera et al., Mol Ther 28:723-746, 2020). Several approaches have been explored to remediate or prevent immune responses; for example, pharmacologic immunosuppressive drugs to block the immune response (Verdera et al., Mol Ther 28:723-746, 2020), vector engineering to decrease the therapeutic dose by maximizing vector potency (Ogden et al., Science 366:1139-1143, 2019), codon optimization to interfere with toll-like receptor (TLR)-target binding (Wright, Mol Ther 28:1756-1758, 2020), and removing preexisting anti-AAV neutralizing antibodies (Leborgne et al., Nat Med 26:1096-1101, 2020; Monteilhet et al., Mol Ther 19:2084-2091, 2011). However, these approaches have their own inherent toxicities (George et al., N Engl J Med 385:1961-1973, 2021) and do not always solve the immune-related toxicities, especially when using high doses of viral vector (High-dose AAV gene therapy deaths, Nat Biotechnol 38:910, 2020). Thus, a need exists for improved AAV gene therapy vectors with reduced immunogenicity and enhanced safety.

### SUMMARY

Disclosed herein is the identification of a promiscuous, immunodominant CD4 T cell epitope in the AAV9 virion protein 1 (VP1), and its elimination in AAV vectors through rational design chimerism. Recombinant AAV vectors engineered to lack the T cell epitope maintained their functions and potency, including yield, cellular specificity, in vitro and in vivo transduction efficacy, and biodistribution in mice, while not eliciting any cellular immune responses. Thus, the present disclosure addresses the unmet need for AAV vectors with reduced immunogenicity and improved safety.

Provided herein are isolated nucleic acid molecules that encode a modified AAV virion VP1 protein lacking a native CD4 T cell epitope. In some aspects, the modified AAV VP1 protein has an amino acid sequence at least 75% identical to the wild type AAV serotype 9 (AAV9) VP1 protein set forth as SEQ ID NO: 1 and includes one or more amino acid substitutions that result in a valine at position 315 and an isoleucine at position 317 of SEQ ID NO: 1; an arginine at position 311, a serine at position 312, an arginine at position 314, a valine at position 315, and an isoleucine at position 317 of SEQ ID NO: 1; an arginine at position 314 of SEQ ID NO: 1; an arginine at position 314 and a valine at position 315 of SEQ ID NO: 1; an arginine at position 314 and an isoleucine at position 317 of SEQ ID NO: 1; or an arginine at position 314, a valine at position 315 and an isoleucine at position 317 of SEQ ID NO: 1. In some examples, the modified VP1 includes a valine at position 315 and an isoleucine at position 317 of SEQ ID NO: 1. In other examples, the modified VP1 includes an arginine at position 311, a serine at position 312, an arginine at position 314, a valine at position 315, and an isoleucine at position 317 of SEQ ID NO: 1.

Also provided are vectors that include a modified VP1-encoding nucleic acid molecule disclosed herein. In some aspects, the vector is an AAV vector, such as an AAV1, AAV2, AAV6, AAV7, AAV8, AAV9, AAV10 or AAV13 vector. In some examples, the AAV vector is an AAV9 vector. In some examples, the AAV vector further includes a heterologous open reading frame (ORF), such as a therapeutic gene.

Host cells that include an isolated nucleic acid molecule or vector disclosed herein are further provided.

Also provided are recombinant AAV vector particles that include a modified AAV VP1 protein lacking a native CD4 T cell epitope. In some aspects, the modified AAV VP1 protein has an amino acid sequence at least 75% identical to the wild type AAV9 VP1 protein set forth as SEQ ID NO: 1 and includes one or more amino acid substitutions that result in a valine at position 315 and an isoleucine at position 317 of SEQ ID NO: 1; an arginine at position 311, a serine at position 312, an arginine at position 314, a valine at position 315, and an isoleucine at position 317 of SEQ ID NO: 1; an arginine at position 314 of SEQ ID NO: 1; an arginine at position 314 and a valine at position 315 of SEQ ID NO: 1; an arginine at position 314 and an isoleucine at position 317 of SEQ ID NO: 1; or an arginine at position 314, a valine at position 315 and an isoleucine at position 317 of SEQ ID NO: 1. In some examples, the modified VP1 includes a valine at position 315 and an isoleucine at position 317 of SEQ ID NO: 1. In other examples, the modified VP1 includes an arginine at position 311, a serine at position 312, an arginine at position 314, a valine at position 315, and an isoleucine at position 317 of SEQ ID NO: 1. The AAV vector particle can be, for example, an AAV1, AAV2, AAV6, AAV7, AAV8, AAV9, AAV10 or AAV13 vector particle. In specific examples, the AAV vector particle further includes an AAV genome, such as a recombinant AAV genome that includes a heterologous ORF (e.g., a therapeutic gene).

Compositions that include a pharmaceutically acceptable carrier and a recombinant AAV vector or a recombinant AAV vector particle disclosed herein are also provided.

Further provided herein is a method of administering a therapeutic gene to a subject by administering to the subject a recombinant AAV vector particle disclosed herein, wherein the vector particle includes a recombinant AAV genome containing the therapeutic gene.

Also provided herein are isolated VP1-derived peptides that include a CD4 T cell epitope. In some aspects, the isolated peptide is no more than 40 amino acids in length and includes the amino acid sequence of SEQ ID NO: 3, SEQ ID NO: 4, SEQ ID NO: 5 or SEQ ID NO: 6. Kits that include an isolated peptide disclosed herein are further provided. Such kits can be used, for example, for immune monitoring assays.

Further provided are methods of inducing immune tolerance against AAV in a subject by administering to the subject an isolated VP1 peptide disclosed herein. In some aspects, the method further includes administering to the subject one or more immunomodulatory agents.

The foregoing and other features of this disclosure will become more apparent from the following detailed description of several aspects which proceeds with reference to the accompanying figures.

### BRIEF DESCRIPTION OF THE DRAWINGS

FIGS. 1A-1G: Rational design of chimeric AAV9 gene therapy vectors. (FIG. 1A) Responses of PBMCs obtained from 52 donors to peptide pools from AAV9 VP1 protein were measured by interleukin (IL)-2 and interferon (IFN)-γ ELISpot assays. PBMCs from healthy donors were stimulated with empty AAV9 capsid for 14 days, followed by restimulation with peptide pools spanning the amino acid sequence of AAV9 VP1. After 24 hours, IL-2 and IFN-γ producing cells were detected by ELISpot assays. A PBMC was considered a responder if the peptides had >3-fold changes on IL-2 or >2.1-fold changes on IFN-γ. (FIG. 1B) Immunodominant peptides (SEQ ID NOs: 3-5) were identified in pool 9. (FIG. 1C) TNF-α and IFN-γ producing cells were measured within the gated CD4 or CD8 T cells using intracellular flow cytometry staining. (FIG. 1D) CD4 or CD8 T cells in AAV9 stimulated PBMCs were depleted by microbeads and stimulated with peptides 103-105 or phytohemagglutinin (PHA). Next, number×size of spots for IFN-γ were analyzed using the ELISpot assay. (FIG. 1E) VP1 peptides 103-105 in AAV serotypes 1 to 13 were aligned using DNAstar software showing a high degree of conservation, except for amino acids R312, L313, N314, F315, and L317. (FIG. 1F) Binding affinity of peptides (SEQ ID NOs: 19-27) to 27 human leukocyte antigen (HLA) class II alleles were predicted using the IEDB T cell epitope prediction tool. Predictions were generated using the IEDB consensus method. The number of alleles were counted if their percentile ranks predicted by the IEDB methods were below 10%. (FIG. 1G) Location of the amino acids which were modified on AAV9 VP1 protein and AAV9 capsid (DiMattia et al., J Virol 86:6947-6958, 2012) (PBD-3UX1, PMID: 22496238). Amino acids K311-V326 that contain the immunodominant epitope and the five amino acids that were modified to the AAV5 sequence are indicated.

FIGS. 2A-2L: The characteristics of chimeric AAV9 variants. (FIGS. 2A-2B) HEK293T were transduced with chimeric AAV9 variants harboring the GFP gene at the indicated multiplicity of infection (MOI; viral genomes (vg)/cell). (FIG. 2A) The percentage of GFP positive cells was determined by flow cytometry. (FIG. 2B) A four parameter curve fit was calculated for each vector and area under the curve (AUC) was calculated. Values are presented as mean±SD (n=3). (FIGS. 2C-2D) The chimeric AAV9 variants expressing NanoLuc were incubated with the indicated concentrations of pooled human serum for 1 hour. HEK293T cells were transduced with the vectors at an MOI of 50000 vg/cell. Transgene expression was determined by luminescence and expressed as transduction efficiency (%). Complete transduction (100%) was defined based on the result of the relative light unit (RLU) obtained from incubation of the AAV vector with fetal bovine serum. Inhibition of vector transduction by neutralizing antibody is expressed as the percentage of transduction. (FIG. 2C) ND50 values were calculated as the dilutions needed to neutralize 50% vector transduction. (FIG. 2D) AUC of FIG. 2C was calculated. Values are presented as mean±SD (n=3). *p<0.05, and **p<0.01. P values were determined by ANOVA with Tukey's multiple comparisons test. (FIGS. 2E-2H) NanoLuc-expressing chimeric AAV9 variants (1×10¹¹ vg/mouse) were injected in Balb/c mice intravenously. (FIG. 2E) Representative image of NanoLuc expression in the mice 8 days after vector administration. (FIG. 2F) Quantification of NanoLuc signal in vector-injected mice on days 8, 17 and 29 days after vector administration. (FIGS. 2G-2H) Representative images (FIG. 2G) and quantification (FIG. 2H) of luciferase signal in various organs of vector-injected mice on day 29. (FIG. 2I) Quantification of viral genome in various organs of vector-injected mice on day 29. (FIG. 2J) PBMCs were stimulated with the indicated AAV vectors for 14 days. Cells were re-stimulated with individual peptide (X-axis; SEQ ID NOs: 19-27 from left to right), and the IFN-γ producing cells were detected by ELISpot assays. Values are presented as mean±SD. (FIGS. 2K-2L) PBMCs were stimulated with the indicated AAV vectors for 14 days. Cells were re-stimulated with individual peptides (SEQ ID NO: 19, SEQ ID NO: 23 and SEQ ID NO: 27 from top to bottom), and the IFN-γ (FIG. 2K) and IL-2 (FIG. 2L) producing cells were measured by ELISpot assays. Fold changes compared to medium control were analyzed (number×size of spots for IFN-γ and number of spots for IL-2). Data are mean±s.e.m. Data were evaluated with a two-tailed unpaired t-test.

FIGS. 3A-3D: Representative pattern of IL-2 and IFN-γ secretion in response to AAV9-derived peptides. (FIGS. 3A-3B) Healthy PBMCs were stimulated with empty AAV9 capsid for 14 days, followed by re-stimulation of AAV9 pools. After 24 hours, IFN-γ (FIG. 3A) and IL-2 (FIG. 3B) producing cells were detected by ELISpot assay. (FIGS. 3C-3D) Responses of IFN-γ (FIG. 3C) and IL-2 (FIG. 3D) to the twelve peptides (97-108) that comprise pool 9. Values are presented as mean±SD

FIGS. 4A-4B: Representative gating strategy. (FIG. 4A) Single cells (FSC-A/FSC-H), FSC-A versus viability dye within the single cells allow detection of live cells. CD8⁺ or CD4⁺ cells within the CD3⁺TCRγδ⁻ population allow the identification of helper CD4 T cells or cytotoxic CD8 T cells. (FIG. 4B) Population of CD4 and CD8 T cells that were analyzed in FIG. 1D.

FIG. 5: Epitope 103-105 is HLA-DP restricted. PBMCs were stimulated with empty AAV9 capsid for 14 days and restimulated with peptide 103-105 in the presence of HLA blocking antibodies. After 24 hours, IFN-γ producing cells were detected by ELISpot assay. The inhibition rates were normalized by isotype control (0%). Each dot represents an individual donor, and each bar shows the mean±SEM.

FIGS. 6A-6C: Comparing IFN-γ secretion in response to AAV5 and AAV9 peptides. (FIG. 6A) A table listing peptides derived from AAV5 or AAV9 that are used in these assays. (FIGS. 6B-6C) Healthy PBMCs were stimulated with empty AAV5 (FIG. 6B) or AAV9 (FIG. 6C) capsid for 14 days, followed by re-stimulation by indicated peptides. After 24 hours, IFN-γ producing cells were detected by ELISpot assay. Fold change of spot number×size was calculated by comparing to respective media only control. Each bar shows the mean±SD.

FIG. 7: Epitopes 103-105 are not found in functionally important amino acid sites. Functionally important amino-acid residues in AAV9 VP1 (SEQ ID NO: 1) are highlighted based on previously published data (Adachi et al., Nat Commun 5:3075, 2014). Black box denotes the epitopes 103-105.

FIGS. 8A-8C: Comparison of the transduction efficacy of chimeric AAV9 variants in other cell lines. HeLa cells (FIGS. 8A and 8C), HEK293T cells (FIG. 8C), and A375 cells (FIGS. 8B and 8C) were transduced with the AAV9, AAV9-VI, AAV9-RSRVI, or AAV5 vectors at the indicated MOI. The percentage of GFP-positive cells was determined by flow cytometry. Each bar shows the mean±SD. *p<0.05, **p<0.01, ***p<0.001 and ****p<0.0001. P values were determined by ANOVA with Tukey's multiple comparisons test. (FIG. 8C) Representative GFP fluorescence images in HeLa, HEK293T, and A375 cells 2 days after transduction of individual AAV vectors. Images were captured at 20× magnification.

FIGS. 9A-9C: Comparison of the transduction efficacy of chimeric AAV9 variants with NanoLuc transgene. HeLa cells (FIG. 9A), HEK293T cells (FIG. 9B), and A375 cells (FIG. 9C) were transduced with the AAV9, AAV9-VI, AAV9-RSRVI, or AAV5 vectors that express NanoLuc at the indicated MOI. The next day, bioluminescence intensities of the cells were determined by luminometer. Each bar shows the mean±SD. *p<0.05, **p<0.01, ***p<0.001 and ****p<0.0001. P values were determined by ANOVA with Tukey's multiple comparisons test.

FIGS. 10A-10B: Neutralizing antibody analysis to chimeric AAV9 variants. The chimeric AAV9 variants expressing NanoLuc was incubated with the indicated concentrations of human serum for 1 hour. HeLa (FIG. 10A) and A375 (FIG. 10B) cells were transduced with the vectors at an MOI of 50,000 and AUC was calculated. Transgene expressions were determined by luminescence and expressed as transduction efficiency (%). Complete transduction (100%) was defined based on the result of the RLU obtained from incubation of the AAV vector with fetal bovine serum. Inhibition of vector transduction by neutralizing antibody is expressed as the percentage of transduction. ND50 values were calculated as the dilutions needed to neutralize 50% vector transduction. Values are presented as mean±SD (n=3). Each bar shows the mean±SD. *p<0.05. P values were determined by ANOVA with Tukey's multiple comparisons test.

FIG. 11: Chimeric AAV9 variants do not create new T-cell epitopes. Representative responses to 20 peptide pools after stimulation with indicated vectors and restimulation with AAV9 peptide pools. Data from two separate experiments were combined.

FIG. 12: Table showing HLA class II alleles for 12 responders.

FIG. 13: Table showing predicted binding of modified AAV9 peptides (SEQ ID NOs: 19-27) to the IEDB major histocompatibility complex (MHC) class II alleles.

FIGS. 14A-14C: The second most prevalent epitope in pool 18 is not good a candidate for chimeric design. (FIGS. 14A-14B) Healthy PBMCs were stimulated with empty AAV9 capsid for 14 days, followed by re-stimulation of individual peptides (205-216) in pool 18 (FIG. 14A) or AAV5 peptides that were aligned to AAV9 peptides (FIG. 14B). After 24 hours, IFN-γ producing cells were detected by ELISpot assay. Fold changes of spot number×size were calculated by comparing to respective media only control. (FIG. 14C) Binding affinity of peptides to 27 HLA class II alleles were predicted using the IEDB T cell epitope prediction tool. Predictions were generated using “the IEDB consensus method”. The number of alleles were counted if their percentile ranks predicted by the IEDB methods were below 10%.

FIG. 15A: Representative silver staining of WT and chimeric AAVs.

FIG. 15B: Representative UV chromatograms displaying the normalized UV intensities (solid line) with overlaid scatter plots showing the Full-Total Ratio (Vg/Cp) for WT and chimeric constructs.

### SEQUENCES

The nucleic acid and amino acid sequences listed in the accompanying sequence listing are shown using standard letter abbreviations for nucleotide bases, and single letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NO: 1 is the amino acid sequence of AAV9 VP1. Residues 307-327,
corresponding to peptides 103-105, are underlined.
MAADGYLPDWLEDNLSEGIREWWALKPGAPQPKANQQHQDNARGLVLPGYKYLGPGNGLDKGEPVNAADAAALEH
DKAYDQQLKAGDNPYLKYNHADAEFQERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGSLTMASGGGAPVADNNEGADGVGSSS
GNWHCDSQWLGDRVITTSTRTWALPTYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDYQLPYVLGSAHEGCLPPFPADVFMIP
QYGYLTLNDGSQAVGRSSFYCLEYFPSQMLRTGNNFQFSYEFENVPFHSSYAHSQSLDRLMNPLIDQYLYYLSKT
INGSGQNQQTLKFSVAGPSNMAVQGRNYIPGPSYRQQRVSTTVTQNNNSEFAWPGASSWALNGRNSLMNPGPAMA
SHKEGEDRFFPLSGSLIFGKQGTGRDNVDADKVMITNEEEIKTTNPVATESYGQVATNHQSAQAQAQTGWVQNQG
ILPGMVWQDRDVYLQGPIWAKIPHTDGNFHPSPLMGGFGMKHPPPQILIKNTPVPADPPTAFNKDKLNSFITQYS
TGQVSVEIEWELQKENSKRWNPEIQYTSNYYKSNNVEFAVNTEGVYSEPRPIGTRYLTRNL
SEQ ID NO: 2 is the amino acid sequence of AAV5 VP1.
MSFVDHPPDWLEEVGEGLREFLGLEAGPPKPKPNQQHQDQARGLVLPGYNYLGPGNGLDRGEPVNRADEVAREHD
ISYNEQLEAGDNPYLKYNHADAEFQEKLADDTSFGGNLGKAVFQAKKRVLEPFGLVEEGAKTAPTGKRIDDHFPK
RKKARTEEDSKPSTSSDAEAGPSGSQQLQIPAQPASSLGADTMSAGGGGPLGDNNQGADGVGNASGDWHCDSTWM
GDRVVTKSTRTWVLPSYNNHQYREIKSGSVDGSNANAYFGYSTPWGYFDFNRFHSHWSPRDWQRLINNYWGFRPR
SLRVKIFNIQVKEVTVQDSTTTIANNLTSTVQVFTDDDYQLPYVVGNGTEGCLPAFPPQVFTLPQYGYATLNRDN
TENPTERSSFFCLEYFPSKMLRTGNNFEFTYNFEEVPFHSSFAPSQNLFKLANPLVDQYLYRFVSTNNTGGVQFN
KNLAGRYANTYKNWFPGPMGRTQGWNLGSGVNRASVSAFATTNRMELEGASYQVPPQPNGMTNNLQGSNTYALEN
TMIFNSQPANPGTTATYLEGNMLITSESETQPVNRVAYNVGGQMATNNQSSTTAPATGTYNLQEIVPGSVWMERD
VYLQGPIWAKIPETGAHFHPSPAMGGFGLKHPPPMMLIKNTPVPGNITSFSDVPVSSFITQYSTGQVTVEMEWEL
KKENSKRWNPEIQYTNNYNDPQFVDFAPDSTGEYRTTRPIGTRYLTRPL
SEQ ID NO: 3 is the amino acid sequence of peptide 103 (GFRPKRLNFKLFNIQ).
SEQ ID NO: 4 is the amino acid sequence of peptide 104 (PKRLNFKLFNIQVKE).
SEQ ID NO: 5 is the amino acid sequence of peptide 105 (LNFKLFNIQVKEVTD).

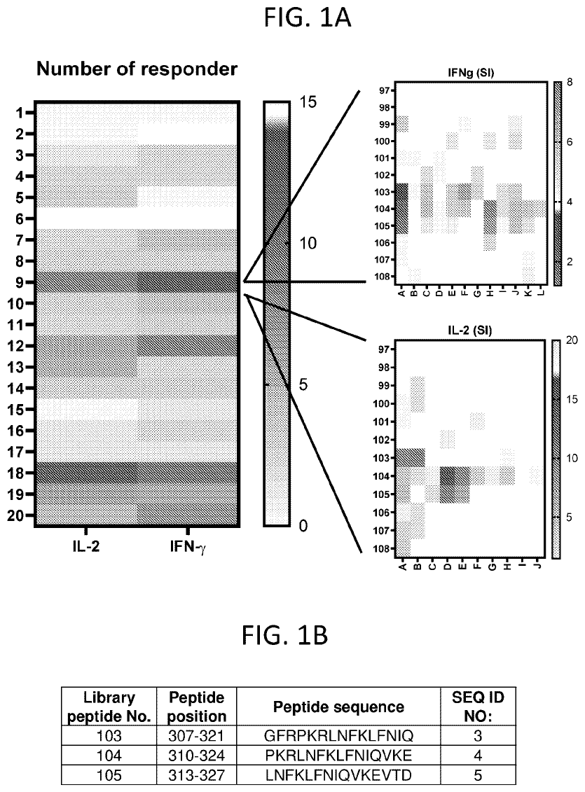

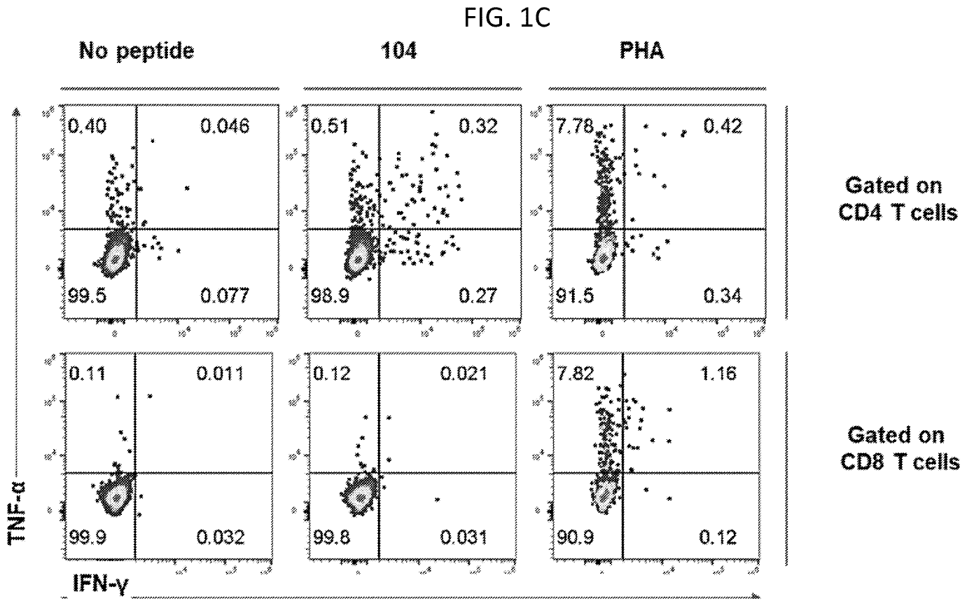

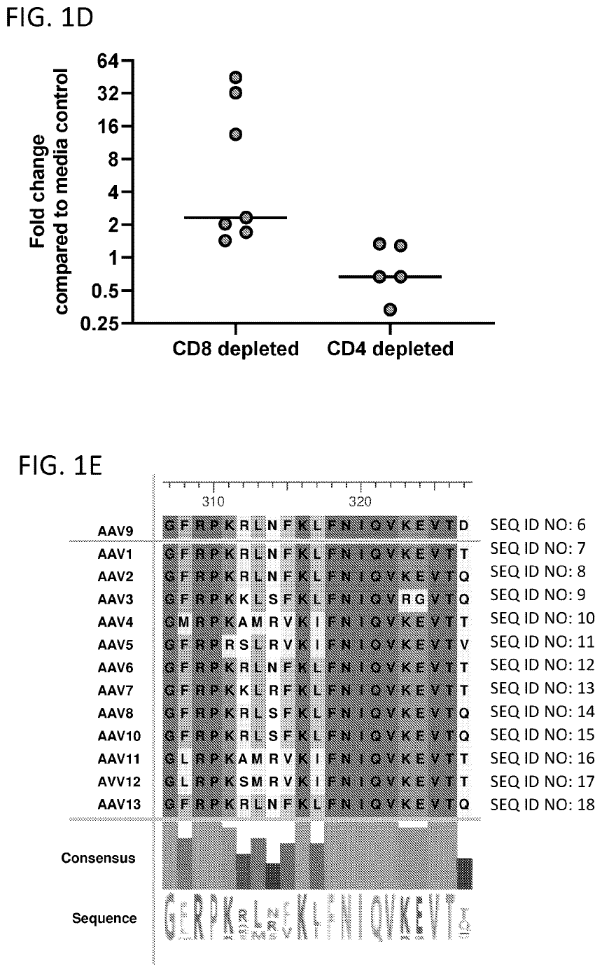

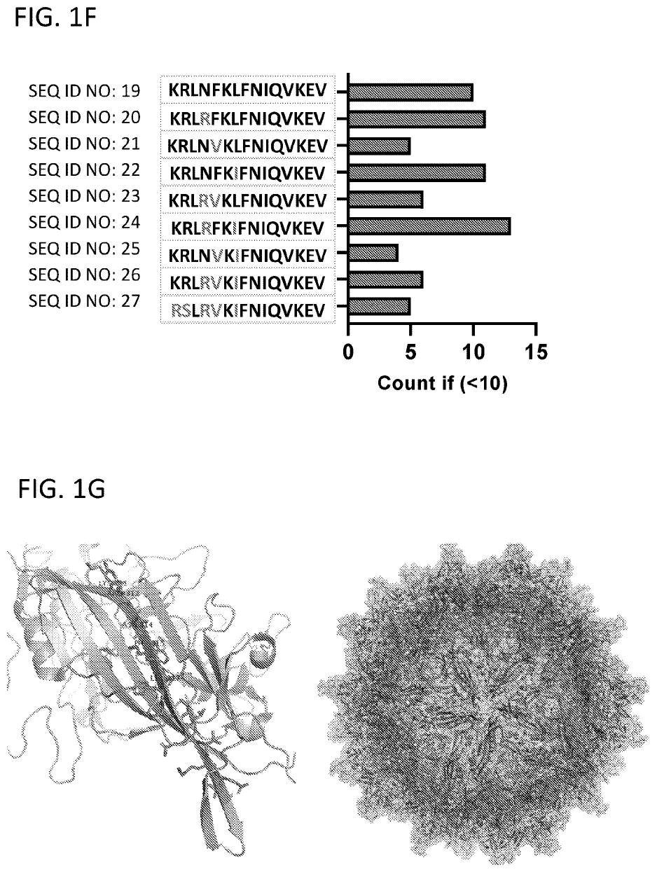

SEQ ID NOs: 6-18 are the amino acid sequences
of the peptides shown in FIG. 1E.

| | | |
|---|---|---|
| AAV9 | GFRPKRLNFKLFNIQVKEVTD | SEQ ID NO: 6 |
| AAV1 | GFRPKRLNFKLFNIQVKEVTT | SEQ ID NO: 7 |
| AAV2 | GFRPKRLNFKLFNIQVKEVTQ | SEQ ID NO: 8 |
| AAV3 | GFRPKKLSFKLFNIQVRGVTQ | SEQ ID NO: 9 |
| AAV4 | GMRPKAMRVKIFNIQVKEVTT | SEQ ID NO: 10 |
| AAV5 | GFRPRSLRVKIFNIQVKEVTV | SEQ ID NO: 11 |
| AAV6 | GFRPKRLNFKLFNIQVKEVTT | SEQ ID NO: 12 |
| AAV7 | GFRPKKLRFKLFNIQVKEVTT | SEQ ID NO: 13 |
| AAV8 | GFRPKRLSFKLFNIQVKEVTQ | SEQ ID NO: 14 |
| AAV10 | GFRPKRLSFKLFNIQVKEVTQ | SEQ ID NO: 15 |
| AAV11 | GLRPKAMRVKIFNIQVKEVTT | SEQ ID NO: 16 |
| AAV12 | GLRPKSMRVIFNIQVKEVTT | SEQ ID NO: 17 |
| AAV13 | GFRPRLNFKLFNIQVEVTQ | SEQ ID NO: 18 |

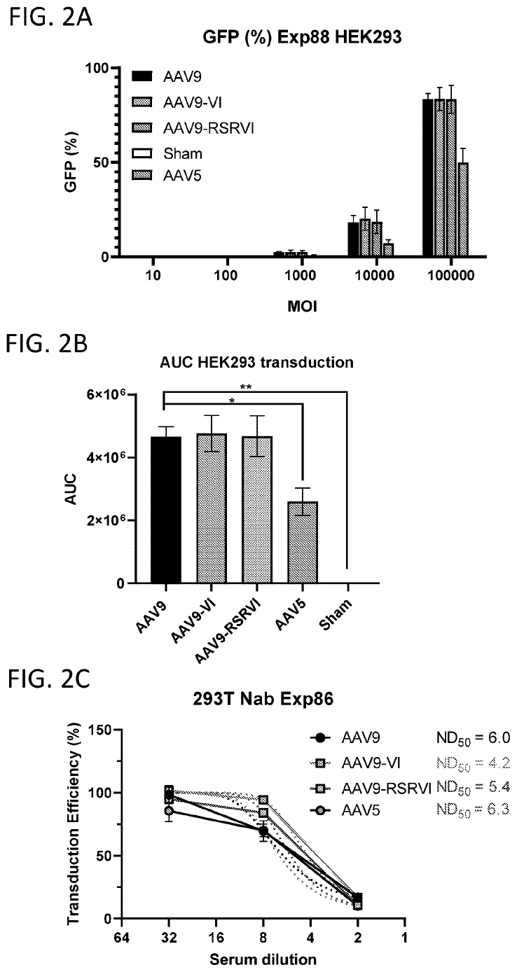

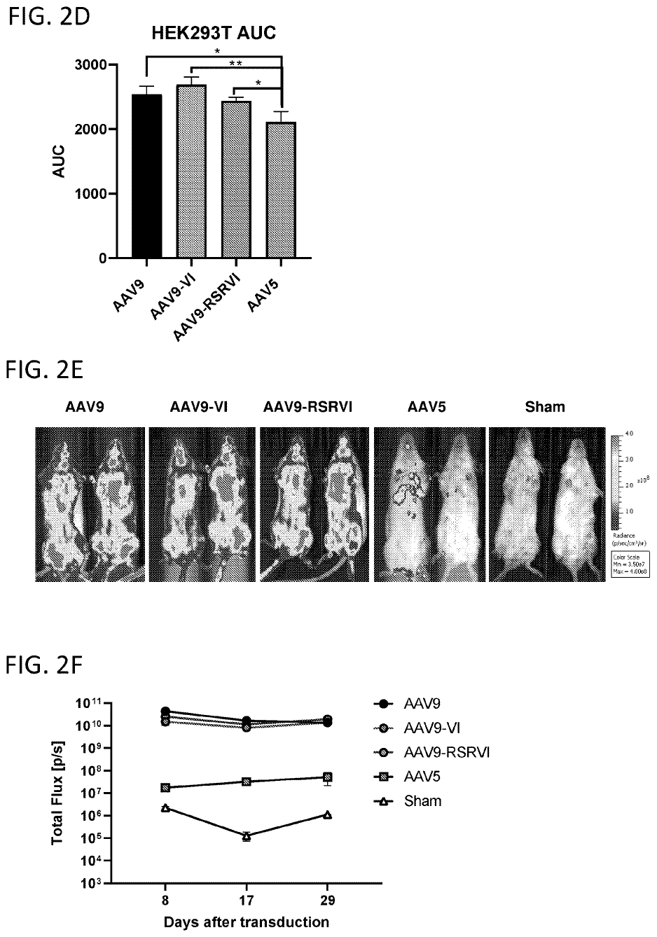

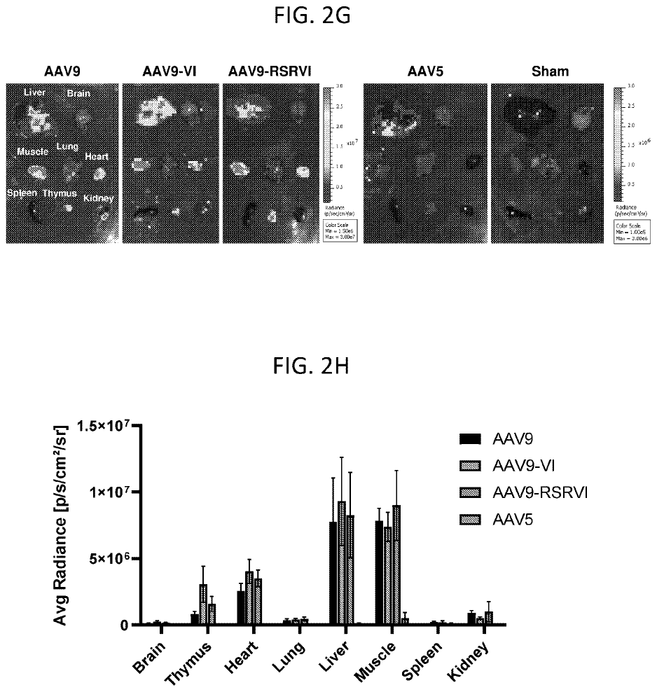

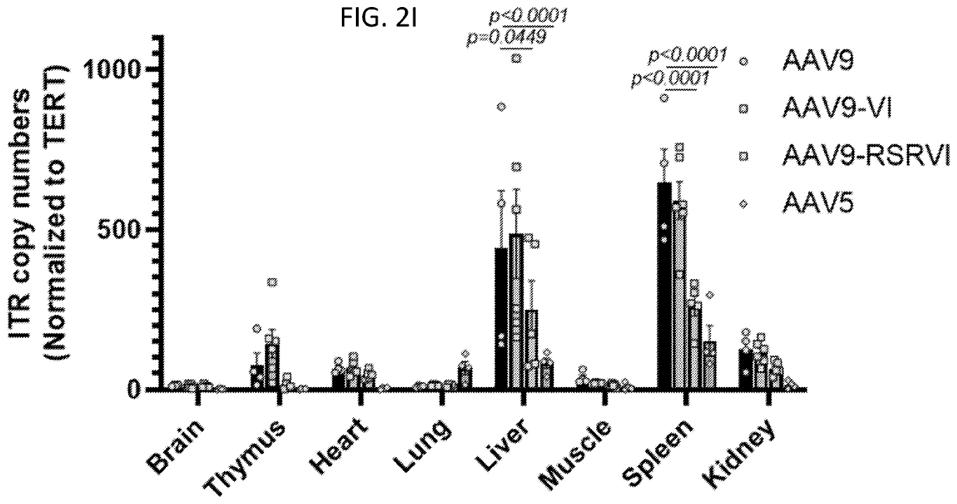

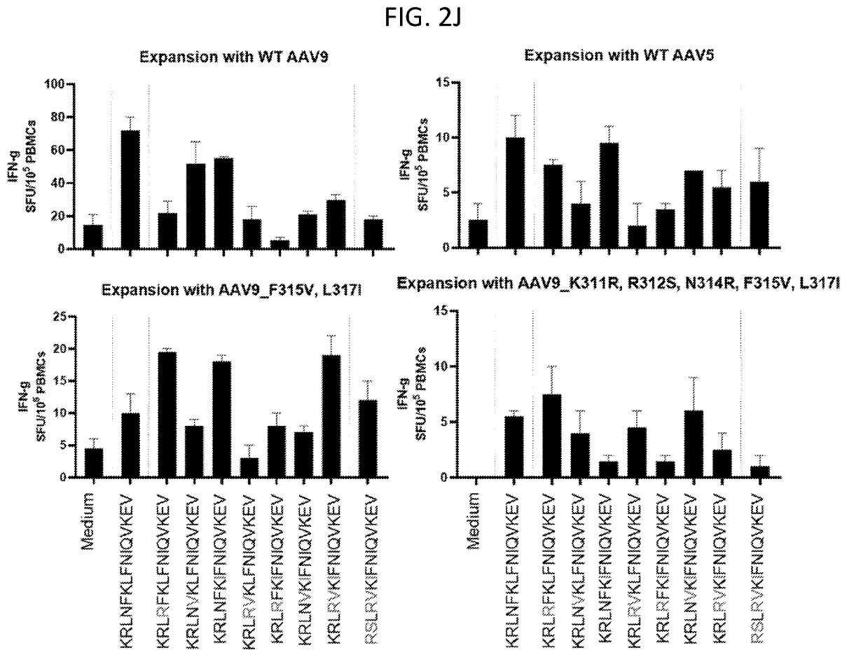

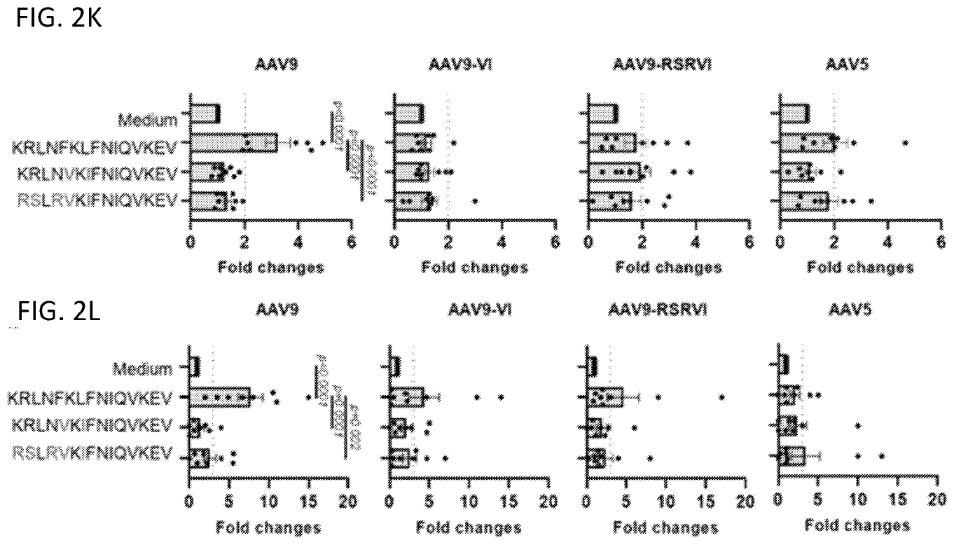

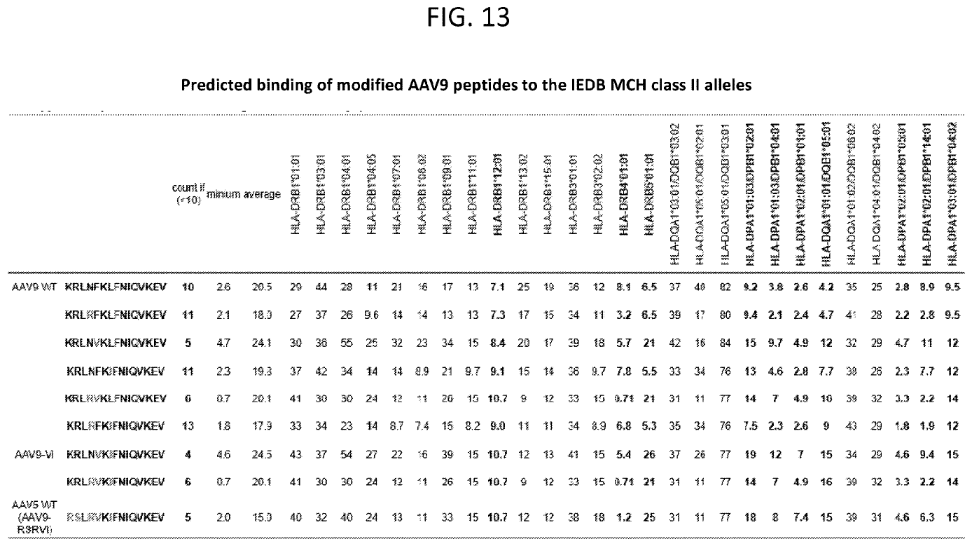

SEQ ID NOs: 19-27 are the amino acid sequences of the peptides shown in FIG. 1F, FIG. 2J and FIG. 13.

SEQ ID NO: 19
KRLNFKLFNIQVKEV
SEQ ID NO: 20
KRLRFKLFNIOVKEV
SEQ ID NO: 21
KRLNVKLFNIQVKEV
SEQ ID NO: 22
KRLNFKIFNIQVKEV
SEQ ID NO: 23
KRLRVKLFNIQVKEV
SEQ ID NO: 24
KRLRFKIFNIQVKEV
SEQ ID NO: 25
KRLNVKIFNIQVKEV
SEQ ID NO: 26
KRLRVKIFNIQVKEV
SEQ ID NO: 27
RSLRVKIFNIQVKEV

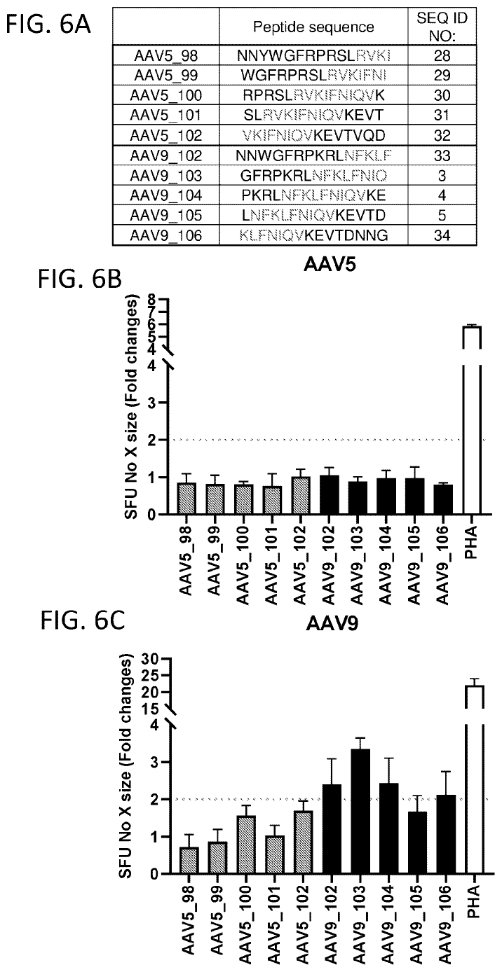

SEQ ID NOs: 3-5 and 28-34 are the amino acid sequences of the peptides shown in FIG. 6A.

AAV5_98
SEQ ID NO: 28
NNYWGFRPRSLRVKI
AAV5_99
SEQ ID NO: 29
WGFRPRSLRVKIFNI
AAV5_100
SEQ ID NO: 30
RPRSLRVKIFNIQVK
AAV5_101
SEQ ID NO: 31
SLRVKIFNIQVKEVT
AAV5_102
SEQ ID NO: 32
VKIFNIQVKEVTVQD
AAV9_102
SEQ ID NO: 33
NNWGFRPKRLNFKLF
AAV9_103
SEQ ID NO: 3
GFRPKRLNFKLFNIQ
AAV9_104
SEQ ID NO: 4
PKRLNFKLFNIQVKE
AAV9_105
SEQ ID NO: 5
LNFKLFNIQVKEVTD
AAV9_106
SEQ ID NO: 34
KLFNIQVKEVTDNNG

SEQ ID NO: 35 is the amino acid sequence of a modified AAV9 VP1 protein with F315V
and L317I substitutions (AAV9-VI; substitutions indicated by bold underline).
MAADGYLPDWLEDNLSEGIREWWALKPGAPQPKANQQHQDNARGLVLPGYKYLGPGNGLDKGEPVNAADAAALEH
DKAYDQQLKAGDNPYLKYNHADAEFQERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGSLTMASGGGAPVADNNEGADGVGSSS
GNWHCDSQWLGDRVITTSTRTWALPTYNNHLYKQISNSTSGGSSNDNAYFGYSTPWGYFDFNRFHCHFSPRDWQR
LINNNWGFRPKRLNVKIFNIQVKEVTDNNGVKTIANNLTSTVQVFTDSDYQLPYVLGSAHEGCLPPFPADVEMIP
QYGYLTLNDGSQAVGRSSFYCLEYFPSQMLRTGNNFQFSYEFENVPFHSSYAHSQSLDRLMNPLIDQYLYYLSKT
INGSGQNQQTLKFSVAGPSNMAVQGRNYIPGPSYRQQRVSTTVTQNNNSEFAWPGASSWALNGRNSLMNPGPAMA
SHKEGEDRFFPLSGSLIFGKQGTGRDNVDADKVMITNEEEIKTTNPVATESYGQVATNHQSAQAQAQTGWVQNQG
ILPGMVWQDRDVYLQGPIWAKIPHTDGNFHPSPLMGGFGMKHPPPQILIKNTPVPADPPTAFNKDKLNSFITQYS
TGQVSVEIEWELQKENSKRWNPEIQYTSNYYKSNNVEFAVNTEGVYSEPRPIGTRYLTRNL
SEQ ID NO: 36 is the amino acid sequence of a modified AAV9 VP1 protein with K311R,
R312S, N314R, F315V, and L317I substitutions (AAV9-RSRVI; substitutions indicated
by bold underline).
MAADGYLPDWLEDNLSEGIREWWALKPGAPQPKANQQHQDNARGLVLPGYKYLGPGNGLDKGEPVNAADAAALEH
DKAYDQQLKAGDNPYLKYNHADAEFQERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGSLTMASGGGAPVADNNEGADGVGSSS
GNWHCDSQWLGDRVITTSTRTWALPTYNNHLYKQISNSTSGGSSNDNAYFGYSTPWGYFDFNRFHCHFSPRDWQR
LINNNWGFRPRSLRVKLINIQVKEVTDNNGVKTIANNLTSTVQVFTDSDYQLPYVLGSAHEGCLPPFPADVEMIP
QYGYLTLNDGSQAVGRSSFYCLEYFPSQMLRTGNNFQFSYEFENVPFHSSYAHSQSLDRLMNPLIDQYLYYLSKT
INGSGQNQQTLKFSVAGPSNMAVQGRNYIPGPSYRQQRVSTTVTQNNNSEFAWPGASSWALNGRNSLMNPGPAMA
SHKEGEDRFFPLSGSLIFGKQGTGRDNVDADKVMITNEEEIKTTNPVATESYGQVATNHQSAQAQAQTGWVQNQG
ILPGMVWQDRDVYLQGPIWAKIPHTDGNFHPSPLMGGFGMKHPPPQILIKNTPVPADPPTAFNKDKLNSFITQYS
TGQVSVEIEWELQKENSKRWNPEIQYTSNYYKSNNVEFAVNTEGVYSEPRPIGTRYLTRNL
SEQ ID NO: 37 is a nucleic acid sequence encoding the AAV9-VI protein
(codon changes relative to WT AAV9 VP1 are indicated by bold underline).
ATGGCTGCCGATGGTTATCTTCCAGATTGGCTCGAGGACAACCTTAGTGAAGGAATTCGCGAGTGGTGGGCTTTG
AAACCTGGAGCCCCTCAACCCAAGGCAAATCAACAACATCAAGACAACGCTCGAGGTCTTGTGCTTCCGGGTTAC
AAATACCTTGGACCCGGCAACGGACTCGACAAGGGGGAGCCGGTCAACGCAGCAGACGCGGCGGCCCTCGAGCAC
GACAAGGCCTACGACCAGCAGCTCAAGGCCGGAGACAACCCGTACCTCAAGTACAACCACGCCGACGCCGAGTTC
CAGGAGCGGCTCAAAGAAGATACGTCTTTTGGGGGCAACCTCGGGCGAGCAGTCTTCCAGGCCAAAAAGAGGCTT
CTTGAACCTCTTGGTCTGGTTGAGGAAGCGGCTAAGACGGCTCCTGGAAAGAAGAGGCCTGTAGAGCAGTCTCCT
CAGGAACCGGACTCCTCCGCGGGTATTGGCAAATCGGGTGCACAGCCCGCTAAAAAGAGACTCAATTTCGGTCAG
ACTGGCGACACAGAGTCAGTCCCAGACCCTCAACCAATCGGAGAACCTCCCGCAGCCCCCTCAGGTGTGGGATCT
CTTACAATGGCTTCAGGTGGTGGCGCACCAGTGGCAGACAATAACGAAGGTGCCGATGGAGTGGGTAGTTCCTCG
GGAAATTGGCATTGCGATTCCCAATGGCTGGGGGACAGAGTCATCACCACCAGCACCCGAACCTGGGCCCTGCCC
ACCTACAACAATCACCTCTACAAGCAAATCTCCAACAGCACATCTGGAGGATCTTCAAATGACAACGCCTACTTC
GGCTACAGCACCCCCTGGGGGTATTTTGACTTCAACAGATTCCACTGCCACTTCTCACCACGTGACTGGCAGCGA
CTCATCAACAACAACTGGGGATTCCGGCCTAAGCGACTCAACGTCAAGATCTTCAACATTCAGGTCAAAGAGGTT
ACGGACAACAATGGAGTCAAGACCATCGCCAATAACCTTACCAGCACGGTCCAGGTCTTCACGGACTCAGACTAT
CAGCTCCCGTACGTGCTCGGGTCGGCTCACGAGGGCTGCCTCCCGCCGTTCCCAGCGGACGTTTTCATGATTCCT
CAGTACGGGTATCTGACGCTTAATGATGGAAGCCAGGCCGTGGGTCGTTCGTCCTTTTACTGCCTGGAATATTTC
CCGTCGCAAATGCTAAGAACGGGTAACAACTTCCAGTTCAGCTACGAGTTTGAGAACGTACCTTTCCATAGCAGC
TACGCTCACAGCCAAAGCCTGGACCGACTAATGAATCCACTCATCGACCAATACTTGTACTATCTCTCAAAGACT
ATTAACGGTTCTGGACAGAATCAACAAACGCTAAAATTCAGTGTGGCCGGACCCAGCAACATGGCTGTCCAGGGA
AGAAACTACATACCTGGACCCAGCTACCGACAACAACGTGTCTCAACCACTGTGACTCAAAACAACAACAGCGAA
TTTGCTTGGCCTGGAGCTTCTTCTTGGGCTCTCAATGGACGTAATAGCTTGATGAATCCTGGACCTGCTATGGCC
AGCCACAAAGAAGGAGAGGACCGTTTCTTTCCTTTGTCTGGATCTTTAATTTTTGGCAAACAAGGAACTGGAAGA
GACAACGTGGATGCGGACAAAGTCATGATAACCAACGAAGAAGAAATTAAAACTACTAACCCGGTAGCAACGGAG
TCCTATGGACAAGTGGCCACAAACCACCAGAGTGCCCAAGCACAGGCGCAGACCGGCTGGGTTCAAAACCAAGGA
ATACTTCCGGGTATGGTTTGGCAGGACAGAGATGTGTACCTGCAAGGACCCATTTGGGCCAAAATTCCTCACACG
GACGGCAACTTTCACCCTTCTCCGCTGATGGGAGGGTTTGGAATGAAGCACCCGCCTCCTCAGATCCTCATCAAA
AACACACCTGTACCTGCGGATCCTCCAACGGCCTTCAACAAGGACAAGCTGAACTCTTTCATCACCCAGTATTCT
ACTGGCCAAGTCAGCGTGGAGATCGAGTGGGAGCTGCAGAAGGAAAACAGCAAGCGCTGGAACCCGGAGATCCAG
TACACTTCCAACTATTACAAGTCTAATAATGTTGAATTTGCTGTTAATACTGAAGGTGTATATAGTGAACCCCGC
CCCATTGGCACCAGATACCTGACTCGTAATCTGTAA
SEQ ID NO: 38 is a nucleic acid sequence encoding the AAV9-RSRVI protein (codon
changes relative to WT AAV9 VP1 are indicated by bold underline).
ATGGCTGCCGATGGTTATCTTCCAGATTGGCTCGAGGACAACCTTAGTGAAGGAATTCGCGAGTGGTGGGCTTTG
AAACCTGGAGCCCCTCAACCCAAGGCAAATCAACAACATCAAGACAACGCTCGAGGTCTTGTGCTTCCGGGTTAC
AAATACCTTGGACCCGGCAACGGACTCGACAAGGGGGAGCCGGTCAACGCAGCAGACGCGGCGGCCCTCGAGCAC
GACAAGGCCTACGACCAGCAGCTCAAGGCCGGAGACAACCCGTACCTCAAGTACAACCACGCCGACGCCGAGTTC
CAGGAGCGGCTCAAAGAAGATACGTCTTTTGGGGGCAACCTCGGGCGAGCAGTCTTCCAGGCCAAAAAGAGGCTT
CTTGAACCTCTTGGTCTGGTTGAGGAAGCGGCTAAGACGGCTCCTGGAAAGAAGAGGCCTGTAGAGCAGTCTCCT
CAGGAACCGGACTCCTCCGCGGGTATTGGCAAATCGGGTGCACAGCCCGCTAAAAAGAGACTCAATTTCGGTCAG
ACTGGCGACACAGAGTCAGTCCCAGACCCTCAACCAATCGGAGAACCTCCCGCAGCCCCCTCAGGTGTGGGATCT
CTTACAATGGCTTCAGGTGGTGGCGCACCAGTGGCAGACAATAACGAAGGTGCCGATGGAGTGGGTAGTTCCTCG
GGAAATTGGCATTGCGATTCCCAATGGCTGGGGGACAGAGTCATCACCACCAGCACCCGAACCTGGGCCCTGCCC
ACCTACAACAATCACCTCTACAAGCAAATCTCCAACAGCACATCTGGAGGATCTTCAAATGACAACGCCTACTTC
GGCTACAGCACCCCCTGGGGGTATTTTGACTTCAACAGATTCCACTGCCACTTCTCACCACGTGACTGGCAGCGA
CTCATCAACAACAACTGGGGATTCCGGCCTCGGTCCCTCAGAGTCAAGATCTTCAACATTCAGGTCAAAGAGGTT
ACGGACAACAATGGAGTCAAGACCATCGCCAATAACCTTACCAGCACGGTCCAGGTCTTCACGGACTCAGACTAT
CAGCTCCCGTACGTGCTCGGGTCGGCTCACGAGGGCTGCCTCCCGCCGTTCCCAGCGGACGTTTTCATGATTCCT
CAGTACGGGTATCTGACGCTTAATGATGGAAGCCAGGCCGTGGGTCGTTCGTCCTTTTACTGCCTGGAATATTTC
CCGTCGCAAATGCTAAGAACGGGTAACAACTTCCAGTTCAGCTACGAGTTTGAGAACGTACCTTTCCATAGCAGC
TACGCTCACAGCCAAAGCCTGGACCGACTAATGAATCCACTCATCGACCAATACTTGTACTATCTCTCAAAGACT
ATTAACGGTTCTGGACAGAATCAACAAACGCTAAAATTCAGTGTGGCCGGACCCAGCAACATGGCTGTCCAGGGA
AGAAACTACATACCTGGACCCAGCTACCGACAACAACGTGTCTCAACCACTGTGACTCAAAACAACAACAGCGAA
TTTGCTTGGCCTGGAGCTTCTTCTTGGGCTCTCAATGGACGTAATAGCTTGATGAATCCTGGACCTGCTATGGCC
AGCCACAAAGAAGGAGAGGACCGTTTCTTTCCTTTGTCTGGATCTTTAATTTTTGGCAAACAAGGAACTGGAAGA
GACAACGTGGATGCGGACAAAGTCATGATAACCAACGAAGAAGAAATTAAAACTACTAACCCGGTAGCAACGGAG
TCCTATGGACAAGTGGCCACAAACCACCAGAGTGCCCAAGCACAGGCGCAGACCGGCTGGGTTCAAAACCAAGGA
ATACTTCCGGGTATGGTTTGGCAGGACAGAGATGTGTACCTGCAAGGACCCATTTGGGCCAAAATTCCTCACACG
GACGGCAACTTTCACCCTTCTCCGCTGATGGGAGGGTTTGGAATGAAGCACCCGCCTCCTCAGATCCTCATCAAA
AACACACCTGTACCTGCGGATCCTCCAACGGCCTTCAACAAGGACAAGCTGAACTCTTTCATCACCCAGTATTCT
ACTGGCCAAGTCAGCGTGGAGATCGAGTGGGAGCTGCAGAAGGAAAACAGCAAGCGCTGGAACCCGGAGATCCAG
TACACTTCCAACTATTACAAGTCTAATAATGTTGAATTTGCTGTTAATACTGAAGGTGTATATAGTGAACCCCGC
CCCATTGGCACCAGATACCTGACTCGTAATCTGTAA

### DETAILED DESCRIPTION

### I. Introduction

The present disclosure describes studies to identify and eliminate immunodominant T cell epitopes within the AAV capsid. Elimination of capsid T cell epitopes enables the development of improved AAV gene therapy vectors that are less immunogenic. In the first clinical trial for hemophilia B treatment, circulating AAV capsid specific T cells increased concurrently with liver enzyme, resulting in transgene expression loss in a few weeks (Mingozzi et al., Nat Med 13:419-422, 2007). The rational design of AAV vectors by replacing components that contain epitopes for T cell recognition has a direct benefit to many gene therapies. To reduce or eliminate immunogenicity of an AAV vector, it is important that the method that eliminates MHC-binding epitopes does not disrupt vector structure and function. Disclosed herein are rationally designed chimeric AAV vectors using integration of ex vivo T cell assays, MHC epitope prediction (Martini et al., Immunogenetics 72:57-76, 2020) and sequence conservation analysis in AAV phylogeny.

II. Abbreviations

| | |
|---|---|
| AAV | adeno-associated virus |
| AUC | area under the curve |
| DLS | dynamic light scattering |
| HLA | human leukocyte antigen |
| IFN | interferon |
| IL | interleukin |
| ITR | inverted terminal repeat |
| MHC | major histocompatibility complex |
| MOI | multiplicity of infection |
| ND50 | 50% neutralization dose |
| ORF | open reading frame |
| PBMC | peripheral blood mononuclear cell |
| PHA | phytohemagglutinin |
| RLU | relative light unit |
| TCR | T cell receptor |
| TLR | toll-like receptor |
| TNF | tumor necrosis factor |
| vg | viral genome |
| VP1 | virion protein 1 |
| WT | wild type |

### III. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “an antigen” includes singular or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

Adeno-associated virus (AAV): A small, replication-defective, non-enveloped virus that infects humans and some other primate species. AAV is not known to cause disease and elicits a mild immune response. Gene therapy vectors that utilize AAV can infect both dividing and quiescent cells and can persist in an extrachromosomal state without integrating into the genome of the host cell. These features make AAV an attractive viral vector for gene therapy. There are currently 13 recognized natural serotypes of AAV (AAV1-13). In the context of the present disclosure, “AAV vector” refers to a nucleic acid AAV-based vector. In some aspects, a disclosed AAV vector includes 5′ and 3′ inverted terminal repeats (ITRs), a heterologous promoter and/or a heterologous ORF (such as a therapeutic gene). In the context of the present disclosure, “AAV vector particle” refers to a viral particle made up of AAV capsid proteins, which include virion protein 1 (VP1), VP2 and VP3. In some aspects of the present disclosure, the AAV vector particle includes a VP1 protein lacking an immunodominant T cell epitope. In some examples, the AAV vector particle further includes an AAV genome, such as a genome including 5′ and 3′ ITRs, a heterologous promoter and/or a heterologous ORF (such as a therapeutic gene).

Administration: To provide or give a subject an agent, such as a therapeutic agent (e.g., a recombinant AAV), by any effective route. Exemplary routes of administration include, but are not limited to, injection (such as subcutaneous, intramuscular, intradermal, intraperitoneal, intravenous, intratumoral, or renal vein injection), oral, intraductal, sublingual, rectal, transdermal, intranasal, vaginal and inhalation routes.

Codon-optimized: A nucleic acid molecule encoding a protein (such as a modified AAV VP1) can be codon-optimized for expression of the protein in a particular organism by including the codon most likely to encode a particular amino acid at each position of the sequence. Codon usage bias is the difference in the frequency of occurrence of synonymous codons (encoding the same amino acid) in coding DNA. A codon is a series of three nucleotides (a triplet) that encodes a specific amino acid residue in a polypeptide chain or for the termination of translation. There are 20 different naturally occurring amino acids, but 64 different codons (61 codons encoding for amino acids plus 3 stop codons). Thus, there is degeneracy because one amino acid can be encoded by more than one codon. A nucleic acid sequence can be optimized for expression in a particular organism (such as a human) by evaluating the codon usage bias in that organism and selecting the codon most likely to encode a particular amino acid. Multivariate statistical methods, such as correspondence analysis and principal component analysis, are widely used to analyze variations in codon usage. Computer programs are available to implement the statistical analyses related to codon usage, such as Codon W, GCUA, and INCA.

Degenerate variant: A polynucleotide encoding a protein (for example, a modified VP1 protein) that includes a sequence that is degenerate as a result of the genetic code. There are twenty natural amino acids, most of which are specified by more than one codon. Therefore, all degenerate nucleotide sequences are included as long as the amino acid sequence of the T cell receptor (TCR), or portion thereof, encoded by the nucleotide sequence is unchanged.

Epitope: An antigenic determinant. These are particular chemical groups or peptide sequences on a molecule that are antigenic (that elicit a specific immune response). A TCR or antibody specifically binds a particular antigenic epitope on a polypeptide.

Heterologous: Originating from a different genetic source. In the context of the present disclosure, a heterologous ORF refers to an ORF that is not native to AAV.

Host cells: Cells in which a vector can be propagated and its nucleic acid expressed. The term also includes any progeny of the subject host cell. It is understood that all progeny may not be identical to the parental cell since there may be mutations that occur during replication. However, such progeny are included when the term “host cell” is used. The cell may be prokaryotic or eukaryotic, such as a mammalian cell, yeast cell, insect cell, or bacterial cell. In some aspects, the cell is a human cell.

Human leukocyte antigen (HLA): Proteins encoded by the MHC gene complex. HLAs from MHC Class I include HLA-A, HLA-B, and HLA-C genes. HLAs from MHC Class II include HLA-DM, HLA-DO, HLA-DP, HLA-DQ, and HLA-DR genes. HLA genes are highly variable, with up to hundreds of variant alleles at some loci.

Immune tolerance: The prevention or inhibition of an immune response against a particular antigen.

Immunomodulatory agent: An agent that stimulates or suppresses the immune system. Immunosuppressive agents can be used to reduce immune responses against foreign antigens, transplanted tissue/organs and to treat some types of autoimmune disease. Exemplary immunosuppressive agents include, for example, cyclosporine A, tacrolimus, sirolimus, prednisone, dexamethasone, azathioprine, cyclophosphamide, and certain types of monoclonal antibodies. Conversely, immunostimulatory agents enhance the immune system, such as for promoting immune responses against infectious agents and tumors. Exemplary immunostimulatory agents include, but are not limited to, BCG, LPS, recombinant cytokines (e.g., IL-2, IL-1, IL-12 and IFN-γ), and antigen-specific antibodies (such as tumor-specific antibodies, such as 3F8, Abagovomab, Adecatumumab, Afutuzumab, Alacizumab, Alemtuzumab, Altumomab pentetate, Anatumomab mafenatox, Apolizumab, Arcitumomab, basiliximab, Bavituximab, Bectumomab, Belimumab, Besilesomab, Bevacizumab, Bivatuzumab mertansine, Blinatumomab, Brentuximab vedotin, Cantuzumab mertansine, Capromab pendetide, Catumaxomab, CC49, Cetuximab, Citatuzumab bogatox, Cixutumumab, Clivatuzumab tetraxetan, Conatumumab, Dacetuzumab, Detumomab, Ecromeximab, Eculizumab, Edrecolomab, Epratuzumab, Ertumaxomab, Etaracizumab, Farletuzumab, Figitumumab, Galiximab, Gemtuzumab ozogamicin, Girentuximab, Glembatumumab vedotin, Ibritumomab tiuxetan, Igovomab, Imciromab, Intetumumab, Inotuzumab ozogamicin, Ipilimumab, Iratumumab, Labetuzumab, Lexatumumab, Lintuzumab, Lorvotuzumab mertansine, Lucatumumab, Lumiliximab, Mapatumumab, Matuzumab, Mepolizumab, Metelimumab, Milatuzumab, Mitumomab, Morolimumab, Nacolomab tafenatox, Naptumomab estafenatox, Necitumumab, Nimotuzumab, Nofetumomab merpentan, Ofatumumab, Olaratumab, Oportuzumab monatox, Oregovomab, Panitumumab, Pemtumomab, Pertuzumab, Pintumomab, Pritumumab, Ramucirumab, Rilotumumab, Rituximab, Robatumumab, Satumomab pendetide, Sibrotuzumab, Sonepcizumab, Tacatuzumab tetraxetan, Taplitumomab paptox, Tenatumomab, TGN1412, Ticilimumab (tremelimumab), Tigatuzumab, TNX-650, Trastuzumab, Tremelimumab, Tucotuzumab celmoleukin, Veltuzumab, Volociximab, Votumumab, and Zalutumumab).

Inverted terminal repeat (ITR): Symmetrical nucleic acid sequences in the genome of adeno-associated viruses required for efficient replication. ITR sequences are located at each end of the AAV DNA genome. The ITRs serve as the origins of replication for viral DNA synthesis and are cis components for generating AAV integrating vectors.

Isolated: An “isolated” biological component (such as a nucleic acid molecule, protein, virus or cell) has been substantially separated or purified away from other biological components in the cell or tissue of the organism, or the organism itself, in which the component occurs, such as other chromosomal and extra-chromosomal DNA and RNA, proteins and cells. Nucleic acid molecules and proteins that have been “isolated” include those purified by standard purification methods. The term also embraces nucleic acid molecules and proteins prepared by recombinant expression in a host cell as well as chemically synthesized nucleic acid molecules and proteins. Isolated does not require absolute purity, and can include proteins, peptides, nucleic acids, viruses, or cells that are at least 50% pure, such as at least 75%, 80%, 90%, 95%, 98%, 99%, or even 99.9% pure.

Open reading frame (ORF): A nucleic acid molecule (e.g., DNA or cDNA) that when translated into amino acids, does not include a stop codon.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carrier: Remington: The Science and Practice of Pharmacy, 22^{nd} ed., London, UK: Pharmaceutical Press, 2013), describes compositions and formulations suitable for pharmaceutical delivery of one or more therapeutic compounds, molecules or agents (e.g., AAV vectors). In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Promoter: A region of DNA that directs/initiates transcription of a nucleic acid (e.g., a gene). A promoter includes necessary nucleic acid sequences near the start site of transcription. Typically, promoters are located near the genes they transcribe. A promoter also optionally includes distal enhancer or repressor elements which can be located as much as several thousand base pairs from the start site of transcription. Promoters can be constitutive, inducible, tissue-specific and/or ubiquitous.

Recombinant: A recombinant nucleic acid molecule is one that has a sequence that is not naturally occurring or has a sequence that is made by an artificial combination of two otherwise separated segments of sequence. This artificial combination can be accomplished by chemical synthesis or by the artificial manipulation of isolated segments of nucleic acid molecules, such as by genetic engineering techniques. Similarly, a recombinant virus is a virus comprising sequence (such as genomic sequence) that is non-naturally occurring or made by artificial combination of at least two sequences of different origin. The term “recombinant” also includes nucleic acids, proteins and viruses that have been altered solely by addition, substitution, or deletion of a portion of a natural nucleic acid molecule, protein, or virus.

Sequence identity: The identity or similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI) and on the internet, for use in connection with the sequence analysis programs blastp, blastn, blastx, tblastn and tblastx. Additional information can be found at the NCBI web site.

Serotype: A group of closely related microorganisms (such as viruses) distinguished by a characteristic set of antigens. AAV has at least 13 known natural serotypes, designated AAV1 to AAV13.

Subject: Living multi-cellular vertebrate organisms, a category that includes human and non-human mammals (such as mice, rats, cats, dogs, rabbits, sheep, horses, cows, goats, pigs, and non-human primates).

Synthetic: Produced by artificial means in a laboratory, for example a synthetic nucleic acid can be chemically synthesized in a laboratory.

Therapeutic gene: A nucleic acid sequence (e.g., a DNA or cDNA sequence) encoding a protein (or functional fragment thereof), or an inhibitory nucleic acid molecule, such as an inhibitory RNA molecule (e.g., a microRNA or short hairpin RNA (shRNA)) useful in the treatment or prevention of a disease, disorder, or condition. For example, a therapeutic gene can inhibit, reduce or eliminate one or more signs or symptoms of a disease, disorder or condition, or can increase survival and/or life expectancy of a subject treated with the therapeutic gene. Therapeutic genes are further described in section VI.

Vector: A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and translation of inserted gene or genes. In some embodiments herein, the vector is an AAV vector.

Virion protein 1 (VP1): One of three capsid proteins of AAV. The AAV cap gene encodes VP1, VP2 and VP3, which assemble to form a protein shell of 60 subunits.

### IV. Recombinant Nucleic Acids, AAV Vectors and AAV Vector Particles

Despite the high safety profile demonstrated in clinical trials, adeno-associated virus (AAV)-mediated gene therapy still faces significant obstacles due to its immunogenicity. To address the unmet need for AAV vectors with reduced immunogenicity, described herein is the identification of a promiscuous, immunodominant CD4 T cell epitope in the AAV VP1 protein, and its elimination through rational design chimerism. Data described herein demonstrates that the recombinant vectors maintained their functions and potency, including yield, cellular specificity, in vitro and in vivo transduction efficacy, and biodistribution in mice, while not eliciting any cellular immune responses.

Disclosed herein are isolated nucleic acid molecules that encode a modified AAV VP1 protein lacking a native CD4 T cell epitope. In some aspects, the modified AAV VP1 protein has an amino acid sequence at least 75% identical to the wild type AAV9 VP1 protein set forth herein as SEQ ID NO: 1 and includes one or more amino acid substitutions that result in a valine at position 315 and an isoleucine at position 317 of SEQ ID NO: 1; an arginine at position 311, a serine at position 312, an arginine at position 314, a valine at position 315, and an isoleucine at position 317 of SEQ ID NO: 1; an arginine at position 314 of SEQ ID NO: 1; an arginine at position 314 and a valine at position 315 of SEQ ID NO: 1; an arginine at position 314 and an isoleucine at position 317 of SEQ ID NO: 1; or an arginine at position 314, a valine at position 315 and an isoleucine at position 317 of SEQ ID NO: 1. In some examples, the modified VP1 includes a valine at position 315 and an isoleucine at position 317 of SEQ ID NO: 1. In other examples, the modified VP1 includes an arginine at position 311, a serine at position 312, an arginine at position 314, a valine at position 315, and an isoleucine at position 317 of SEQ ID NO: 1. For VP1 proteins from AAV serotypes having a native VP1 protein of a different length than AAV9 VP1, reference to positions 311, 312, 314, 315 and 317 means the positions that correspond to positions 311, 312, 314, 315 and 317 of SEQ ID NO: 1.

In some aspects, the AAV is AAV1, AAV2, AAV6, AAV7, AAV8, AAV9, AAV10 or AAV13. In some examples, the AAV is AAV9. In other examples, the AAV vector is a hybrid of two or more AAV serotypes (such as, but not limited to, AAV2/1, AAV2/7, AAV2/8 or AAV2/9). The selection of AAV serotype will depend in part on the cell type(s) that are targeted for gene therapy.

In some aspects, the amino acid sequence of the modified VP1 protein is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 1, and has a valine at position 315 and an isoleucine at position 317 (and may also have an arginine at position 311, a serine at position 312, and/or an arginine at position 314, such as 1, 2 or all 3 of these) all with reference to SEQ ID NO: 1. In some examples, the amino acid sequence of the modified VP1 protein includes or consists of SEQ ID NO: 35 (AAV9-VI) or SEQ ID NO: 36 (AAV9-RSRVI).

In other aspects, the amino acid sequence of the modified VP1 protein is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 1, and has an arginine at position 314 of SEQ ID NO: 1; an arginine at position 314 and a valine at position 315 of SEQ ID NO: 1; an arginine at position 314 and an isoleucine at position 317 of SEQ ID NO: 1; or an arginine at position 314, a valine at position 315 and an isoleucine at position 317 of SEQ ID NO: 1.

In some aspects, the nucleic acid molecule is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 37 or SEQ ID NO: 38, and encodes a valine at position 315 and an isoleucine at position 317 (and may also encode an arginine at position 311, a serine at position 312, and/or an arginine at position 314, such as 1, 2 or all 3 of these) all with reference to SEQ ID NO: 1. In some examples, the nucleic acid molecule includes or consists of the sequence of SEQ ID NO: 37 (or a degenerate variant thereof) or SEQ ID NO: 38 (or a degenerate variant thereof).

In some aspects, the nucleic acid sequence is codon-optimized for expression in mammalian cells, such as in human cells, dog cells, pig cells, cat cells, or non-human primate cells.

Also disclosed herein are vectors that include an isolated nucleic acid molecule described herein. In some aspects, the isolated nucleic acid molecule is operably linked to a promoter. In some examples, the promoter is a tissue-specific promoter. Exemplary tissue-specific promoters include, for example, thyroxin binding globulin (TBG) promoter, insulin promoter, glucagon promoter, somatostatin promoter, mucin-2 promoter, pancreatic polypeptide (PPY) promoter, synapsin-1 (Syn) promoter, retinoschisin promoter, K12 promoter, CC10 promoter, surfactant protein C (SP-C) promoter, PRC1 promoter, RRM2 promoter, uroplakin 2 (UPII) promoter, or lactoferrin promoter. In other examples, the promoter is a constitutive promoter, such as the Rous sarcoma virus LTR promoter, cytomegalovirus (CMV) promoter, SV40 promoter, dihydrofolate reductase promoter, β-actin promoter, phosphoglycerol kinase (PGK) promoter or EF1α promoter. In other examples, the promoter is an inducible promoter. Examples of inducible promoters include the zinc-inducible sheep metallothionine (MT) promoter, the dexamethasone (Dex)-inducible mouse mammary tumor virus (MMTV) promoter, the T7 polymerase promoter system, the tetracycline-repressible system, the tetracycline-inducible system, the RU486-inducible system, and the rapamycin-inducible system. In some instances, the vector further includes other regulatory sequences, such as one or more enhancers.

In some aspects, the vector is an AAV vector, such as an AAV1, AAV2, AAV3, AAV6, AAV7, AAV8, AAV9, AAV10 or AAV13 vector. In some examples, the AAV vector is an AAV9 vector. In other examples, the AAV vector is a hybrid of two or more AAV serotypes (such as, but not limited to, AAV2/1, AAV2/7, AAV2/8 or AAV2/9). In some examples, the AAV vector includes 5′ and 3′ ITRs. In some examples, the AAV vector further includes a heterologous open reading frame (ORF), such as a therapeutic gene. The therapeutic gene can be any nucleic acid sequence (such as a DNA or cDNA sequence) encoding a protein or an inhibitory nucleic acid molecule (such as an inhibitory RNA molecule, e.g., a microRNA or shRNA) that is useful in the treatment or prevention of a disease, disorder, or condition. Non-limiting examples of therapeutic genes are provided in section VI.

Further described herein are isolated host cells that include a nucleic acid molecule or vector disclosed herein. In some aspects, the isolated host cell is a cell (or cell line) appropriate for production of recombinant AAV. In some examples, the host cell is a mammalian cell, such as a HEK-293, BHK, Vero, RD, HT-1080, A549, Cos-7, ARPE-19, or MRC-5 cell. In particular examples, the cells are 293 cells or a derivative thereof (e.g., FREESTYLE 293-F cells).

Also provided herein are recombinant AAV vector particles that include a nucleic acid molecule encoding a VP1 protein as disclosed herein.

Further provided herein are recombinant AAV vector particles that include a modified VP1 protein having an amino acid sequence at least 75% identical to SEQ ID NO: 1, wherein the modified VP1 protein has includes one or more amino acid substitutions that result in a valine at position 315 and an isoleucine at position 317 of SEQ ID NO: 1; an arginine at position 311, a serine at position 312, an arginine at position 314, a valine at position 315, and an isoleucine at position 317 of SEQ ID NO: 1; an arginine at position 314 of SEQ ID NO: 1; an arginine at position 314 and a valine at position 315 of SEQ ID NO: 1; an arginine at position 314 and an isoleucine at position 317 of SEQ ID NO: 1; or an arginine at position 314, a valine at position 315 and an isoleucine at position 317 of SEQ ID NO: 1. In some examples, the modified VP1 includes a valine at position 315 and an isoleucine at position 317 of SEQ ID NO: 1. In other examples, the modified VP1 includes an arginine at position 311, a serine at position 312, an arginine at position 314, a valine at position 315, and an isoleucine at position 317 of SEQ ID NO: 1. For VP1 proteins from AAV serotypes having a native VP1 protein of a different length than AAV9 VP1, reference to positions 311, 312, 314, 315 and 317 means the positions that correspond to positions 311, 312, 314, 315 and 317 of SEQ ID NO: 1.

In some aspects, the AAV is AAV1, AAV2, AAV6, AAV7, AAV8, AAV9, AAV10 or AAV13. In some examples, the AAV is AAV9. In other examples, the AAV vector is a hybrid of two or more AAV serotypes (such as, but not limited to, AAV2/1, AAV2/7, AAV2/8 or AAV2/9). The selection of AAV serotype will depend in part on the cell type(s) that are targeted for gene therapy.

In some aspects, the amino acid sequence of the modified VP1 protein is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 1, and has a valine at position 315 and an isoleucine at position 317 (and may also have an arginine at position 311, a serine at position 312, and/or an arginine at position 314, such as 1, 2 or all 3 of these) all with reference to SEQ ID NO: 1. In some examples, the amino acid sequence of the modified VP1 protein includes or consists of SEQ ID NO: 35 (AAV9-VI) or SEQ ID NO: 36 (AAV9-RSRVI).

In other aspects, the amino acid sequence of the modified VP1 protein is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 1, and has an arginine at position 314 of SEQ ID NO: 1; an arginine at position 314 and a valine at position 315 of SEQ ID NO: 1; an arginine at position 314 and an isoleucine at position 317 of SEQ ID NO: 1; or an arginine at position 314, a valine at position 315 and an isoleucine at position 317 of SEQ ID NO: 1.

In some aspects, the recombinant AAV vector particle further includes an AAV genome. In some examples, the AAV genome includes 5′ and 3′ ITRs. In some examples, the AAV vector further includes a heterologous ORF, such as a therapeutic gene. The therapeutic gene can be any nucleic acid sequence (such as a DNA or cDNA sequence) encoding a protein or an inhibitory nucleic acid molecule (such as an inhibitory RNA molecule, e.g., a microRNA or shRNA) useful in the treatment or prevention of a disease, disorder, or condition. Non-limiting examples of therapeutic genes are provided in section VI.

Further provided are methods of administering a therapeutic gene to a subject by administering to the subject a recombinant AAV vector particle containing an AAV genome in which the therapeutic gene is inserted, as disclosed herein. In some aspects, the therapeutic gene is a therapeutic gene listed in section VI. In some aspects, the recombinant AAV vector particles are administered at a dose of about 1×10¹⁰ to about 1×10¹⁴ viral particles (vp)/kg. In some examples, the AAV vector particles are administered at a dose of about 1×10¹¹ to about 1×10¹³ vp/kg or at a dose of about 1×10¹² vp/kg. The recombinant AAV vector particles can be administered in a single dose, or in multiple doses (such as 2, 3, 4, 5, 6, 7, 8, 9 or 10 doses) as needed for the desired therapeutic results.

Also provided are compositions that include a disclosed recombinant AAV vector particle and a pharmaceutically acceptable carrier, such as water or saline. In some examples, the composition is a liquid, frozen, or lyophilized. In some aspects, the compositions are formulated for intravenous, transdermal, intraocular, intrathecal, intracerebral, oral, subcutaneous, intranasal, inhalation or intramuscular administration. Suitable pharmaceutical formulations for administration of AAV can be found, for example, in U.S. Patent Application Publication No. 2012/0219528.

Further provided are isolated peptides that contain the immunodominant CD4 T cell epitope (or a portion thereof) disclosed herein. In some aspects the isolated peptide is no more than 40 amino acids in length and includes the amino acid sequence of SEQ ID NO: 3, SEQ ID NO: 4, SEQ ID NO: 5 or SEQ ID NO: 6. In some examples, the peptide is no more than 39, no more than 38, no more than 37, nor more than 36, no more than 35, no more than 34, no more than 33, no more than 32, no more than 31, no more than 30, no more than 29, no more than 28, no more than 27, no more than 26, no more than 25, no more than 24, no more than 23, no more than 22 or no more than 21 amino acids in length. In some examples, the isolated peptide is 21-40, 25-40, 30-40, or 35-40 amino acids in length. In particular examples, the amino acid sequence of the peptide consists of SEQ ID NO: 3, SEQ ID NO: 4, SEQ ID NO: 5 or SEQ ID NO: 6.

Further provided are methods of inducing immune tolerance against AAV in a subject. In some aspects, the method includes administering to the subject one or more of the isolated peptides disclosed herein. The peptides can be administered in multiple doses, such as 2, 3, 4 or 5 doses. In some examples, the method further includes administering to the subject one or more immunomodulatory agents. In particular examples, the immunomodulatory agent is cyclosporine A, tacrolimus, sirolimus, prednisone, dexamethasone, azathioprine, cyclophosphamide, or a monoclonal antibody. In some examples, the peptide(s) is/are encapsulated in a nanoparticle or a microparticle.

Also provided herein are kits that include one or more isolated peptides disclosed herein. Such kits can be used, for example, for immune monitoring assays. In some examples, a kit includes one or more recombinant AAV vector particles disclosed herein, for example for use with the therapeutic methods provided herein. In some aspects, the kit further includes solid support(s), buffer(s), syringe(s), container(s) and/or instructional material(s). The instructional materials may be written, in an electronic form or may be visual (such as video files).

### V. Recombinant AAV for Gene Therapy Applications

AAV belongs to the family Parvoviridae and the genus Dependovirus. AAV is a small, non-enveloped virus that packages a linear, single-stranded DNA genome. Both sense and antisense strands of AAV DNA are packaged into AAV capsids with equal frequency.

The AAV genome is characterized by two inverted terminal repeats (ITRs) that flank two open reading frames (ORFs). In the AAV2 genome, for example, the first 125 nucleotides of the ITR are a palindrome, which folds upon itself to maximize base pairing and forms a T-shaped hairpin structure. The other 20 bases of the ITR, called the D sequence, remain unpaired. The ITRs are cis-acting sequences important for AAV DNA replication; the ITR is the origin of replication and serves as a primer for second-strand synthesis by DNA polymerase. The double-stranded DNA formed during this synthesis, which is called replicating-form monomer, is used for a second round of self-priming replication and forms a replicating-form dimer. These double-stranded intermediates are processed via a strand displacement mechanism, resulting in single-stranded DNA used for packaging and double-stranded DNA used for transcription. Located within the ITR are the Rep binding elements and a terminal resolution site (TRS). These features are used by the viral regulatory protein Rep during AAV replication to process the double-stranded intermediates. In addition to their role in AAV replication, the ITR is also needed for AAV genome packaging, transcription, negative regulation under non-permissive conditions, and site-specific integration (Daya and Berns, Clin Microbiol Rev 21(4):583-593, 2008).

The left ORF of AAV contains the Rep gene, which encodes four proteins—Rep78, Rep 68, Rep52 and Rep40. The right ORF contains the Cap gene, which produces three viral capsid proteins (VP1, VP2 and VP3). The AAV capsid contains 60 viral capsid proteins arranged into an icosahedral symmetry. VP1, VP2 and VP3 are present in a 1:1:10 molar ratio (Daya and Berns, Clin Microbiol Rev 21(4):583-593, 2008).

AAV is a frequently used virus for gene therapy. Although AAV infects humans and some other primate species, it is not known to cause disease and elicits a mild immune response. Gene therapy vectors that utilize AAV can infect both dividing and quiescent cells and persist in an extrachromosomal state without integrating into the genome of the host cell.

AAV possesses several desirable features for a gene therapy vector, including the ability to bind and enter target cells, enter the nucleus, the ability to be expressed in the nucleus for a prolonged period of time, and relatively low toxicity. However, the small size of the AAV genome limits the size of heterologous DNA that can be incorporated. To minimize this obstacle, AAV vectors have been constructed that do not encode Rep and the integration efficiency element (IEE). The ITRs are retained as they are cis signals needed for packaging (Daya and Berns, Clin Microbiol Rev 21(4):583-593, 2008).

Methods for producing rAAV suitable for gene therapy are well known (see, for example, U.S. Patent Application Nos. 2012/0100606; 2012/0135515; 2011/0229971; and 2013/0072548; U.S. Pat. No. 11,578,340; and Ghosh et al., Gene Ther 13(4):321-329, 2006), and can be utilized with the recombinant nucleic acid molecules, vectors and methods disclosed herein.

### VI. Therapeutic Genes

The recombinant AAV vectors disclosed herein can optionally include a heterologous ORF, such as a therapeutic gene, for use in gene therapy. The therapeutic gene can encode any protein (or functional fragment thereof) that is useful in the treatment, inhibition, gene editing, or prevention of a disease, disorder or condition. Alternatively, the therapeutic gene can encode an inhibitory nucleic acid molecule, such as an inhibitory RNA (e.g., a microRNA or shRNA). Non-limiting examples of therapeutic genes that can be used in the AAV vectors and vector particles disclosed herein are listed below (see also U.S. Pat. No. 11,578,340).

In some aspects, the therapeutic gene encodes a growth factor, interleukin, interferon, anti-apoptosis factor, cytokine, anti-diabetic factor, anti-apoptosis agent, coagulation factor, or anti-tumor factor. In some examples, the therapeutic gene is the BDNF, CNTF, CSF, EGF, FGF, G-SCF, GM-CSF, gonadotropin, IFN, IFG-1, M-CSF, NGF, PDGF, PEDF, TGF, VEGF, TGF-B2, TNF, prolactin, somatotropin, XIAP1, IL-1, IL-2, IL-3, IL-4, IL-5, IL-6, IL-7, IL-8, IL-9, IL-10, IL-10 (187A), viral IL-10, IL-11, IL-12, IL-13, IL-14, IL-15, IL-16 IL-17, or IL-18 gene. In some examples, the therapeutic gene encodes an inhibitory nucleic acid molecule that modulates (such as inhibits) expression of one of the above-listed genes.

In some aspects, the therapeutic gene is a gene for treating a disease associated with the reduced expression, loss of expression or dysfunctional expression of a gene. Such genes include genes encoding, for example, glucose-6-phosphatase, phosphoenolpyruvate-carboxykinase, galactose-1 phosphate uridyl transferase, phenylalanine hydroxylase, branched chain alpha-ketoacid dehydrogenase, fumarylacetoacetate hydrolase, methylmalonyl-CoA mutase, medium chain acyl CoA dehydrogenase, ornithine transcarbamylase, argininosuccinic acid synthetase, low density lipoprotein receptor protein, UDP-glucouronosyltransferase, adenosine deaminase, hypoxanthine guanine phosphoribosyl transferase, biotinidase, beta-glucocerebrosidase, beta-glucuronidase, peroxisome membrane protein 70 kDa, porphobilinogen deaminase, alpha-1 antitrypsin, erythropoietin vascular endothelial growth factor, angiopoietin-1, fibroblast growth factor, thrombomodulin, tissue factor pathway inhibitor, aromatic amino acid decarboxylase (AADC), tyrosine hydroxylase (TH), beta adrenergic receptor, sarco(endo)plasmic reticulum adenosine triphosphatase-2 (SERCA2), cardiac adenylyl cyclase, p53, dystrophin, minidystrophin, utrophin, miniutrophin, and insulin. In some examples, the therapeutic gene encodes an inhibitory nucleic acid molecule that modulates (such as inhibits) expression of one of the above-listed genes.

In some aspects, the therapeutic gene is useful in the treatment of a disease, condition or disorder associated with the central nervous system. In some examples, the therapeutic gene is DRD2, GRIA1, GRIA2, GRIN1, SLC1A1, SYP, SYT1, CHRNA7, 3 Rtau/4 rTUS, APP, BAX, BCL-2, GRIK1, GFAP, IL-1, AGER, UCH-L1, SKP1, EGLN1, Nurr-1, BDNF, TrkB, gstm1, S106β, IT15, PRNP, JPH3, TBP, ATXN1, ATXN2, ATXN3, Atrophin 1, FTL, TITF-1, FXN, ASPA, DMD, SMN1, UBE1, or DYNC1H1.

In some aspects, the therapeutic gene is a gene useful in the treatment of a disease, disorder or condition associated with the cardiovascular system. In some examples, the gene is VEGF, FGF, SDF-1, connexin 40, connexin 43, SCN4a, HIF1α, SERCa2a, ADCY1, or ADCY6. In some examples, the therapeutic gene encodes an inhibitory nucleic acid molecule that modulates (such as inhibits) expression of one of the above-listed genes.

In some aspects, the therapeutic gene is a gene useful in the treatment of a disease, disorder or condition associated with the pulmonary system. In some examples, the gene is TNFα, TGFβ1, SFTPA1, SFTPA2, SFTPB, SFTPC, HPS1, HPS3, HPS4, ADTB3A, IL1A, IL1B, LTA, IL6, CXCR1, or CXCR2. In some examples, the therapeutic gene encodes an inhibitory nucleic acid molecule that modulates (such as inhibits) expression of one of the above-listed genes.

In some aspects, the therapeutic gene is a gene useful in the treatment of a disease, disorder or condition associated with the liver (e.g., α1-AT, HFE, ATP7B, fumarylacetoacetate hydrolase (FAH), glucose-6-phosphatase, NCAN, GCKR, LYPLAL1, or PNPLA3), kidney (e.g., PKD1, PKD2, PKHD1, NPHS1, NPHS2, PLCE1, CD2AP, LAMB2, TRPC6, WT1, LMX1B, SMARCAL1, COQ2, PDSS2, SCARB3, FN1, COL4A5, COL4A6, COL4A3, COL4A4, FOX1C, RET, UPK3A, BMP4, SIX2, CDC5L, USF2, ROBO2, SLIT2, EYA1, MYOG, SIX1, SIX5, FRAS1, FREM2, GATA3, KAL1, PAX2, TCF2, or SALL1), eye (e.g., CFH, C3, MT-ND2, ARMS2, TIMP3, CAMK4, FMN1, RHO, USH2A, RPGR, RP2, TMCO, SIX1, SIX6, LRP12, ZFPM2, TBK1, GALC, myocilin, CYP1B1, CAV1, CAV2, optineurin or CDKN2B), gastrointestinal tract (e.g., CYP2C19, CCL26, APC, IL12, IL10, or IL-18), pancreas (e.g., PRSS1, SPINK1, STK11, MLH1, KRAS2, p16, p53, or BRAF), urinary tract (e.g., HSPA1B, CXCR1 & 2, TLR2, TLR4, TGF-1, FGFR3, RB1, HRAS, TP53, or TSC1), or uterus (e.g., DN-ER, MLH1, MSH2, MSH6, PMS1, or PMS). In some examples, the therapeutic gene encodes an inhibitory nucleic acid molecule that modulates (such as inhibits) expression of one of the above-listed genes.

In some aspects, the therapeutic gene is a gene useful in the treatment of a cancer. In some examples, the gene is AARS, ABCB1, ABCC4, ABI2, ABL1, ABL2, ACK1, ACP2, ACY1, ADSL, AK1, AKR1C2, AKT1, ALB, ANPEP, ANXA5, ANXA7, AP2M1, APC, ARHGAP5, ARHGEF5, ARID4A, ASNS, ATF4, ATM, ATP5B, ATP50, AXL, BARD1, BAX, BCL2, BHLHB2, BLMH, BRAF, BRCA1, BRCA2, BTK, CANX, CAP1, CAPN1, CAPNS1, CAV1, CBFB, CBLB, CCL2, CCND1, CCND2, CCND3, CCNE1, CCT5, CCYR61, CD24, CD44, CD59, CDC20, CDC25, CDC25A, CDC25B, CDC2L5, CDK10, CDK4, CDK5, CDK9, CDKL1, CDKN1A, CDKN1B, CDKN1C, CDKN2A, CDKN2B, CDKN2D, CEBPG, CENPC1, CGRRF1, CHAF1A, CIB1, CKMT1, CLK1, CLK2, CLK3, CLNS1A, CLTC, COL1A1, COL6A3, COX6C, COX7A2, CRAT, CRHR1, CSF1R, CSK, CSNK1G2, CTNNA1, CTNNB1, CTPS, CTSC, CTSD, CUL1, CYR61, DCC, DCN, DDX10, DEK, DHCR7, DHRS2, DHX8, DLG3, DVL1, DVL3, E2F1, E2F3, E2F5, EGFR, EGR1, EIF5, EPHA2, ERBB2, ERBB3, ERBB4, ERCC3, ETV1, ETV3, ETV6, F2R, FASTK, FBN1, FBN2, FES, FGFR1, FGR, FKBP8, FN1, FOS, FOSL1, FOSL2, FOXG1A, FOXO1A, FRAP1, FRZB, FTL, FZD2, FZD5, FZD9, G22P1, GAS6, GCN5L2, GDF15, GNA13, GNAS, GNB2, GNB2L1, GPR39, GRB2, GSK3A, GSPT1, GTF2I, HDAC1, HDGF, HER2, HMMR, HPRT1, HRB, HSPA4, HSPA5, HSPA8, HSPB1, HSPH1, HYAL1, HYOU1, ICAM1, ID1, ID2, IDUA, IER3, IFITM1, IGF1R, IGF2R, IGFBP3, IGFBP4, IGFBP5, IL1B, ILK, ING1, IRF3, ITGA3, ITGA6, ITGB4, JAK1, JARID1A, JUN, JUNB, JUND, K-ALPHA-1, KIT, KITLG, KLK10, KPNA2, KRAS2, KRT18, KRT2A, KRT9, LAMB1, LAMP2, LCK, LCN2, LEP, LITAF, LRPAP1, LTF, LYN, LZTR1, MADH1, MAP2K2, MAP3K8, MAPK12, MAPK13, MAPKAPK3, MAPRE1, MARS, MAS1, MCC, MCM2, MCM4, MDM2, MDM4, MET, MGST1, MICB, MLLT3, MME, MMP1, MMP14, MMP17, MMP2, MNDA, MSH2, MSH6, MT3, MYB, MYBL1, MYBL2, MYC, MYCL1, MYCN, MYD88, MYL9, MYLK, NEO1, NF1, NF2, NFKB1, NFKB2, NFSF7, NID, NINJ1, NMBR, NME1, NME2, NME3, NOTCH1, NOTCH2, NOTCH4, NPM1, NQO1, NR1D1, NR2F1, NR2F6, NRAS, NRG1, NSEP1, OSM, PA2G4, PABPC1, PARP1, PCNA, PCTK1, PCTK2, PCTK3, PDGFA, PDGFB, PDGFRA, PDPK1, PEA15, PFDN4, PFDN5, PGAM1, PHB, PIK3CA, PIK3CB, PIK3CG, PIM1, PKM2, PKMYT1, PLK2, PPARD, PPARG, PPIH, PPP1CA, PPP2R5A, PRDX2, PRDX4, PRKAR1A, PRKCBP1, PRNP, PRSS15, PSMA1, PTCH, PTEN, PTGS1, PTMA, PTN, PTPRN, RAB5A, RAC1, RAD50, RAF1, RALBP1, RAP1A, RARA, RARB, RASGRF1, RB1, RBBP4, RBL2, REA, REL, RELA, RELB, RET, RFC2, RGS19, RHOA, RHOB, RHOC, RHOD, RIPK1, RPN2, RPS6KB1, RRM1, SARS, SELENBP1, SEMA3C, SEMA4D, SEPP1, SERPINH1, SFN, SFPQ, SFRS7, SHB, SHH, SIAH2, SIVA, SIVA TP53, SKI, SKIL, SLC16A1, SLC1A4, SLC20A1, SMO, SMPD1, SNAI2, SND1, SNRPB2, SOCS1, SOCS3, SOD1, SORT1, SPINT2, SPRY2, SRC, SRPX, STAT1, STAT2, STAT3, STAT5B, STC1, TAF1, TBL3, TBRG4, TCF1, TCF7L2, TFAP2C, TFDP1, TFDP2, TGFA, TGFB1, TGFB1, TGFBR2, TGFBR3, THBS1, TIE, TIMP1, TIMP3, TJP1, TK1, TLE1, TNF, TNFRSF10A, TNFRSF10B, TNFRSF1A, TNFRSF1B, TNFRSF6, TNFSF7, TNK1, TOB1, TP53, TP53BP2, TP53I3, TP73, TPBG, TPT1, TRADD, TRAM1, TRRAP, TSG101, TUFM, TXNRD1, TYRO3, UBC, UBE2L6, UCHL1, USP7, VDAC1, VEGF, VHL, VIL2, WEE1, WNT1, WNT2, WNT2B, WNT3, WNT5A, WT1, XRCC1, YES1, YWHAB, YWHAZ, ZAP70, or ZNF9. In some examples, the therapeutic gene encodes an inhibitory nucleic acid molecule that modulates (such as inhibits) expression of one of the above-listed genes.

In some aspects, the therapeutic gene that modulates apoptosis, such as RPS27A, ABL1, AKT1, APAF1, BAD, BAG1, BAG3, BAG4, BAK1, BAX, BCL10, BCL2, BCL2A1, BCL2L1, BCL2L10, BCL2L11, BCL2L12, BCL2L13, BCL2L2, BCLAF1, BFAR, BID, BIK, NAIP, BIRC2, BIRC3, XIAP, BIRC5, BIRC6, BIRC7, BIRC8, BNIP1, BNIP2, BNIP3, BNIP3L, BOK, BRAF, CARD10, CARD11, NLRC4, CARD14, NOD2, NOD1, CARD6, CARDS, CARDS, CASP1, CASP10, CASP14, CASP2, CASP3, CASP4, CASP5, CASP6, CASP7, CASP8, CASP9, CFLAR, CIDEA, CIDEB, CRADD, DAPK1, DAPK2, DFFA, DFFB, FADD, GADD45A, GDNF, HRK, IGF1R, LTA, LTBR, MCL1, NOL3, PYCARD, RIPK1, RIPK2, TNF, TNFRSF10A, TNFRSF10B, TNFRSF10C, TNFRSF10D, TNFRSF11B, TNFRSF12A, TNFRSF14, TNFRSF19, TNFRSF1A, TNFRSF1B, TNFRSF21, TNFRSF25, CD40, FAS, TNFRSF6B, CD27, TNFRSF9, TNFSF10, TNFSF14, TNFSF18, CD40LG, FASLG, CD70, TNFSF8, TNFSF9, TP53, TP53BP2, TP73, TP63, TRADD, TRAF1, TRAF2, TRAF3, TRAF4, TRAF5 DRD2, GRIA1, GRIA2, GRIN1, SLC1A1, SYP, SYT1, CHRNA7, 3 Rtau/4 rTUS, APP, BAX, BCL-2, GRIK1, GFAP, IL-1, AGER, UCH-L1, SKP1, EGLN1, Nurr-1, BDNF, TrkB, gstm1, S106β, IT15, PRNP, JPH3, TBP, ATXN1, ATXN2, ATXN3, Atrophin 1, FTL, TITF-1, FXN, ASPA, DMD, and SMN1, UBE1, or DYNC1H1. In some examples, the therapeutic gene encodes an inhibitory nucleic acid molecule that modulates (such as inhibits) expression of one of the above-listed genes.

### EXAMPLES

The following examples are provided to illustrate particular features of certain aspects of the disclosure, but the scope of the claims should not be limited to those features exemplified.

### Example 1: Materials and Methods

This example describes the materials and experimental procedures used for the studies described in Example 2.

### Plasmid and DNA Mutagenesis

For production of rAAV, plasmids pAAV2/9n (112865, Addgene, Watertown, MA), pAAV2/5n (104964, Addgene), pHelper vector (340202, Cell Biolabs, San Diego, CA), pscAAV-GFP (AAV-410, Cell Biolabs), and pAAV-CMV-NanoLuc-Halotag (Promega, Madison, WI) were used. Empty capsids were produced by transfecting pHelper and pAAV2/9n or pAAV2/5n. Plasmid mutagenesis was performed by Gene Universal (Newark, DE). Accuracy of the nucleotide changes were verified by DNA sequence analyses immediately after production and again after plasmid expansion.

### Recombinant Vector Production

Empty AAV9 and AAV5 capsids were produced using a slightly modified protocol described by Kimura et al. (Zincarelli et al., Mol Ther 16:1073-1080, 2008). Briefly, Free Style 293F cells (Thermo Fisher Scientific, San Jose, CA) were transfected using PEI MAX (Polysciences, Warrington, PA) and lysed in an acidic buffer, the homogenates were cleared from debris by centrifugation, and the pH was neutralized using HEPES buffer. Later, the AAV particles in the lysates and medium were recovered by polyethylene glycol (PEG) precipitation.

GFP-expressing and NanoLuc-expressing AAVs were generated using the standard triple transfection method (Bing et al., Mol Ther Methods Clin Dev 24:255-267, 2022). Briefly, Viral Production Cells 2.0 (Thermo Fisher Scientific) were transfected and harvested 72 hours thereafter. After three freeze-thaw cycles, sonication and BENZONASE™ treatment, AAVs were purified using two successive ultracentrifugation rounds in iodixanol gradients. For sham control, all procedures were identical except for the presence of the Rep/Cap plasmid. The capsid protein in the vectors was quantified using an AAV5 or AAV9 ELISA kit (Progen, Wayne, PA). The copy titers of vector genomes were determined using Taqman quantitative PCR (qPCR) as previously described (Bing et al., Mol Ther Methods Clin Dev 24:255-267, 2022).

### Peptide Synthesis

A total of 242 15-mer peptides overlapping 12 amino acids as well as chimeric and AAV5 VP1 derived peptides were purchased from GenScript Biotech (Piscataway, NJ). The peptides cover the entire sequence of VP1 from the AAV9 capsid. The purity of peptides was >95%, as determined by high-performance liquid chromatography. Lyophilized peptides were dissolved in dimethyl sulfoxide (DMSO) at 50 mg/mL and diluted in RPMI media. For the initial screening, peptides were pooled into groups of 12 consecutive peptides, except for pools 19 and 20, which each contained 13 peptides.

### Human PBMC Samples

Peripheral mononuclear cells (PBMCs) were collected from apheresis samples of 52 healthy donors. Samples were isolated using gradient-density separation by Ficoll-Hypaque (GE Healthcare, Chicago, IL) according to the manufacturer's instructions, and cryopreserved in liquid nitrogen until assayed. HLA typing was performed as described previously by Scisco Genetics (Seattle, WA) (Puig et al., Front Immunol 11:629399, 2020).

### In Vitro Expansion of AAV-Specific PBMC Cells

PBMCs were thawed and resuspended at a concentration of 5×10⁶ cells/mL in RPMI media containing 5% heat-inactivated human serum, 1% GLUTAMAX™ (Thermo Fisher Scientific), 1 mM sodium pyruvate (Thermo Fisher Scientific), 10 mM HEPES (Thermo Fisher Scientific), MEM Non-Essential Amino Acids (Thermo Fisher Scientific), and 1% penicillin/streptomycin (Thermo Fisher Scientific). PBMCs were stimulated with heated empty AAV9 capsid, empty AAV5 capsid, or mutated AAVs (2×10¹⁰ vp/mL). Next, cells were supplemented with fresh assay medium containing 20 units of IL-2 (MilliporeSigma, Burlington, MA), 5 μg/mL of IL-7 (Biolegend, San Diego, CA), and 25 μg/mL of IL-15 (Biolegend) every 3-4 days after initial antigenic stimulation. On day 14, cells were harvested and screened for reactivity against AAV-specific peptide pools or individual peptides.

### ELISpot Assay

The secretion of IL-2 and IFN-γ was analyzed using an ELISpot assay according to the manufacturer's recommendations (Mabtech, Cincinnati, OH). After in vitro expansion, cells were harvested, and incubated at a density of 100,000 cells/well either with peptide pools or individual peptides (10 μg/mL) in plates pre-coated with anti-human IL-2 or IFN-γ antibodies. Negative controls were treated with medium, and positive controls were treated with CEF, CEFT (PANATecs, Baden-Wuerttemberg, Germany) or PHA (MilliporeSigma). After 24 hours, spots were developed with biotin-conjugated anti-IL-2 or anti-IFN-γ antibody (Mabtech, Nacka Strand, Sweden), streptavidin alkaline phosphate (Mabtech), and nitro blue tetrazolium and 5-bromo-4-chloro-3′-indolyl phosphate (BCIP/NBT) substrate (KPL, Thermo Fisher Scientific). Computer software (Immunospot 7.0; Cellular Technology Limited, Cleveland, OH) was used to enumerate spots forming cells (SFC). For IFN-γ analysis, spot size was incorporated into the analysis by multiplying the spot counts by the average spot size per well. The responses were considered positive if the magnitude of the responses exceeded 2.1 (IFN-γ) or 3 (IL-2) times the background level of the negative controls.

For experiments utilizing depletion of CD4 or CD8 T cells, CD4 T cells were purified by negative selection using magnetic beads and LD columns (Miltenyi Biotec, Bergisch Gladbach, Germany), and CD8 T cells were isolated by positive selection using magnetic beads and LS columns (Miltenyi Biotec) according to the manufacturer's protocol.

For the HLA restriction assay, expanded PBMC with AAVs were incubated with 20 μg/mL monoclonal Abs against HLA Class I (W6/32), HLA-DR (G46-6), HLA-DQ (SPV-L3), or HLA-DP (B7/21) 30 minutes prior to peptide addition. Cytokine production against positive peptides was then measured by ELISpot assay as described above.

### FACS

After in vitro expansion, cells were restimulated with 10 μg/mL peptides for 24 hours at 37 °C. Cytokine secretion in cell cultures was blocked by the addition of GolgiPlug/GolgiStop (BD Biosciences, San Jose, CA) for 5 hours prior to cell harvesting and staining. Cells were stained for surface markers CD3 (clone UCHT1), CD4 (clone SK3), CD8 (clone RPA-T8), CD56 (clone HCD56), and TCRγδ (clone 11F2). After washing, cells were fixed and permeabilized using CYTOFIX/CYTOPERM solution (BD Biosciences) and then stained for IFN-γ (clone B27), and TNF-α (clone MAb11). Acquisition was performed using a Cytek Aurora (Cytek Biosciences, Fremont, CA), and analysis was performed using the Flowjo (Treestar, Ashland, OR) software.

### In Silico HLA Binding Prediction

Peptide binding affinities for HLA class I and class II were predicted using the binding prediction tool available at the Immune Epitope Database (IEDB) (online at iedb.org; Reynisson et al., Nucleic Acids Res 48:W449-w454, 2020; Wang et al., BMC Bioinformatics 11:568, 2010). HLA binding ranking was predicted for each 9-mer peptide with 27 HLA class I alleles (Weiskopf et al., Proc Natl Acad Sci USA 110:E2046-2053, 2013), and 15-mer peptide with 27 HLA class II alleles (Wang et al., BMC Bioinformatics 11:568, 2010). These alleles represent binding specificities that are shared by the majority of the world's population (Wang et al., BMC Bioinformatics 11:568, 2010; Weiskopf et al., Proc Natl Acad Sci USA 110:E2046-2053, 2013). Percentile rank offers a consistent scale that enables comparisons between various predictors. Higher affinities are indicated by a lower percentile rank value. Potential binders in AAV9 VP1 were identified using an initial cutoff of percentile ranks less than 1% or 10% for HLA class I or class II allele, respectively.

### In Vitro Transduction and Neutralization Assay

HeLa, HEK293T or A375 cells were seeded in 96-well plates at a final density of 2×10⁴ cells/well in DMEM (Thermo Fisher) media containing 10% fetal bovine serum (FBS, Millipore sigma), and penicillin/streptomycin. After 24 hours, media was changed to DMEM+2% FBS media and virus was added at MOIs of 10, 100, 1000, 10000, and 100000 vg/cell. At 48-hour post transduction, the expression of GFP was analyzed by direct fluorescence imaging and flow cytometry. To detect NanoLuc luciferase, furimazine (Promega) was added to the culture at 24 hours after transduction, and the results were immediately assessed by luminometer.

A neutralization assay was performed as described previously (Meliani et al., Hum Gene Ther Methods 26:45-53, 2015). Briefly, serial dilutions of pooled heat-inactivated human serum (MilliporeSigma) were mixed with vectors expressing GFP or NanoLuc and incubated for one hour. After incubation, samples were added to cells and reporter genes were measured after 24 hours.

### Dynamic Light Scattering (DLS)

The AAV particles were diluted to a concentration of 2×10¹¹ vg/ml in resuspension buffer based on qPCR values and filtered using a 0.22 microfilter. Each well was loaded with 30 μL solution into a 384-well microtiter plate (Aurora) in triplicates. DLS data were collected in a DynaPro Plate Reader (Waters|Wyatt Technology, Santa Barbara, CA). For thermal stability measurements, the temperature was ramped discretely from 4 °C to 85 °C at a rate of 1.0 °C/min, and Rh was measured throughout the temperature ramp. Data acquisition and analysis were performed with DYNAMICS® software with a solvent viscosity set at 5% Glycerol.

### SEC-MALS

Size-exclusion separations were performed using an Agilent 1260 Infinity II high performance liquid chromatography system (HPLC) system (Agilent, Santa Clara, CA). The system consists of a binary pump, multisampler maintained at 4 °C, multicolumn thermostat maintained at 25 °C, and diode array detector. A DAWN® multiangle light scattering (MALS) detector and OPTILAB® differential refractive index (RI) detector (Waters|Wyatt Technology) were attached in series with the HPLC. The MALS detector was fitted with an internal dynamic light scattering fiber for simultaneous in-line DLS measurements (detection angle of 135°). The system was equipped with a XBridge Premier GTx BEH size exclusion column (7.8 mm×30 cm, 2.5 μm particle size, 450 Å pore size, Waters Corporation, Milford, MA). The mobile phase used for all injections was 10 mM sodium phosphate, 350 mM sodium chloride, 0.001 vol/vol % Pluronic® F-68, pH 7.4. Flow rate was maintained at 0.5 m/min for all injections. Injections were performed using VISION® 3.2.0.67 (Waters|Wyatt Technology). MALS, UV, and RI data were collected and analyzed using ASTRA® 8.1.2.1 (Waters|Wyatt Technology).

### In Vivo Bioluminescence Imaging of NanoLuc

6-8-week-old BALB/c mice were purchased from The Jackson Laboratory (Bar Harbor, ME). NanoLuc expressing rAAV vectors were diluted in 200 μL of PBS before being injected intravenously through the tail vein at a dose of 10¹¹ vg/mouse (4-6 mice/group). On days 8, 17, and 29, mice were injected with 0.44 μmol of fluorofurimazine (Promega), anesthetized with isoflurane, and imaged after 3±1 minutes using an IVIS spectrum imager (PerkinElmer, Waltham, MA). On day 29, mouse organs were extracted and rinsed in PBS before being analyzed with an IVIS imager. By subtracting the signal from the same organ of the sham controls, signal quantification in specific regions of interest (ROIs) were corrected for background.

### Example 2: Identification of Immunodominant Epitopes in the AAV9 Capsid

To identify the immunodominant epitopes in the AAV9 capsid, 242 overlapping peptides that span the sequence of AAV9 VP1, and human PBMCs from a cohort of 52 donors with a distribution of HLA alleles comparable to that in the North American population, were used. The peptides were put into pools, each containing 12 overlapping peptides. Only pools that tested positive on the IL-2 and IFN-γ ELISpot assays were deconvoluted to determine which specific peptides contained the epitopes.

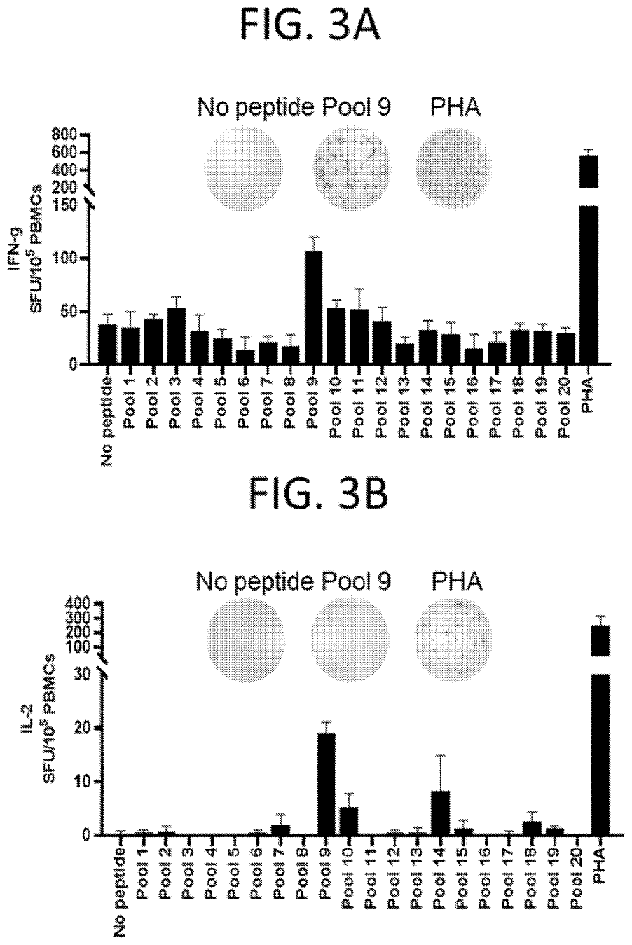

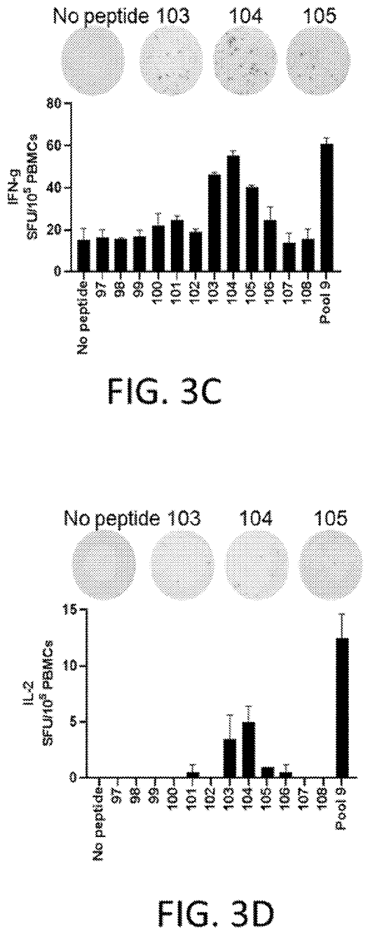

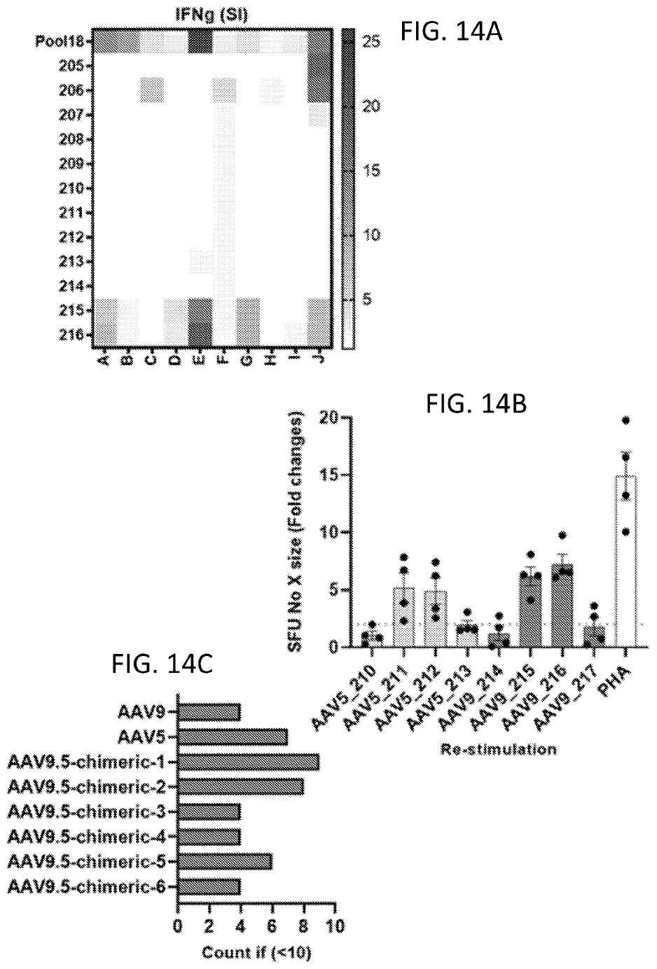

FIG. 1A shows heat maps of the responses of the 52 donors. IL-2 and IFN-γ response rates to pool 9 were 17% and 23%, respectively; and to pool 18, 23% and 19%, respectively. Stimulation with the individual peptides that constitute these pools (peptides 97-108 in pool 9 and peptides 205-216 in pool 18) revealed that pool 9 contained most immunodominant epitope (peptide 103-105) in AAV9 capsid protein (FIG. 1A and FIGS. 3A-3D) while pool 18 contained the second and third most immunodominant epitopes (FIG. 14A and Table 2). The immunodominant epitope in AAV9 was identified as containing between 307 and 327 amino acids (FIG. 1B) and immune response to this epitope was found in 23% (12 of 52) of donor samples (Table 1).

TABLE 1
IL-2 and IFN-γ secretion in response
to peptide 103-105 in 12 donors

| | IL-2 | | | IFN-γ | | |
|---|---|---|---|---|---|---|
| | SFU/10⁵ cells | | | (SFU/10⁵ cells) × size | | |
| | | Peptide | | | Peptide | |
| Donor | No peptide | 103-105 | Fold | No peptide | 103-105 | Fold |
| #4 | 6.0 | 22.0 | 3.7 | 285.2 | 416.1 | 1.5 |
| #9 | 1.7 | 5.5 | 3.2 | 435.2 | 945.2 | 2.2 |
| #17 | 1.0 | 10.0 | 10.0 | 1112.0 | 2266.1 | 2.0 |
| #18 | 1.3 | 28.3 | 21.8 | 631.0 | 1262.5 | 2.0 |
| #22 | 19.7 | 154.0 | 7.8 | 2584.2 | 5235.3 | 2.0 |
| #46 | 2.0 | 7.0 | 3.5 | 212.1 | 393.3 | 1.9 |
| #47 | 3.7 | 8.7 | 2.4 | 415.4 | 1095.4 | 2.6 |
| #49 | 0.8 | 5.0 | 6.7 | 264.6 | 1497.8 | 5.7 |
| #59 | 3.7 | 11.7 | 3.2 | 2961.5 | 5931.5 | 2.0 |
| #73 | 0.7 | 4.0 | 5.7 | 273.0 | 551.6 | 2.0 |
| #106 | 1.0 | 2.3 | 2.3 | 131.5 | 319.9 | 2.4 |
| #107 | 1.0 | 10.3 | 10.3 | 544.1 | 1106.5 | 2.0 |

TABLE 2
Top 3 T cell epitopes in AAV9 VP1

| | | Number of | | | | | | | |
|---|---|---|---|---|---|---|---|---|---|
| | | responders | | | HLA I binding³ | | HLA II binding³ | | |
| | | (total n = 52)¹ | | Reacted | | Median | count | Median | |
| Peptide | Pool | IL-2 | IFN-γ | cell | count | percentile | if | percentile | HLA |
| Number | Number | ELISpot | ELISpot | types² | if <1 | rank | <10 | rank | Restriction |
| 103, 104, 105 | 9 | 11 | 10 | CD4 | 5 | 5.8 | 10 | 16 | DP |
| 215, 216 | 18 | 9 | 7 | CD4 | 1 | 14 | 4 | 33 | DR |
| 205, 206 | 18 | 8 | 6 | CD4 | 5 | 5.1 | 0 | 61 | DR |

| | | | | |
|---|---|---|---|---|
| penton protein in Adenovirus | 6 | 3.4 | | |
| pseudomonas exotoxin | | | 7 | 22 |

¹Responder was defined experimentally if the peptides had >3 fold changes on IL-2 SFC/1E5 or >2 fold changes on IFN-γ SFC/1E5.
²Reacted T cell types is defined by intracellular flow cytometry, if the number of IL-2, IFN-γ, or TNF-α producing cells were more than 1.5 fold changes compared to media only controls, and confirmed by ELISpot assay.
³HLA binding predictions were using IEDB class I and class II prediction tools.

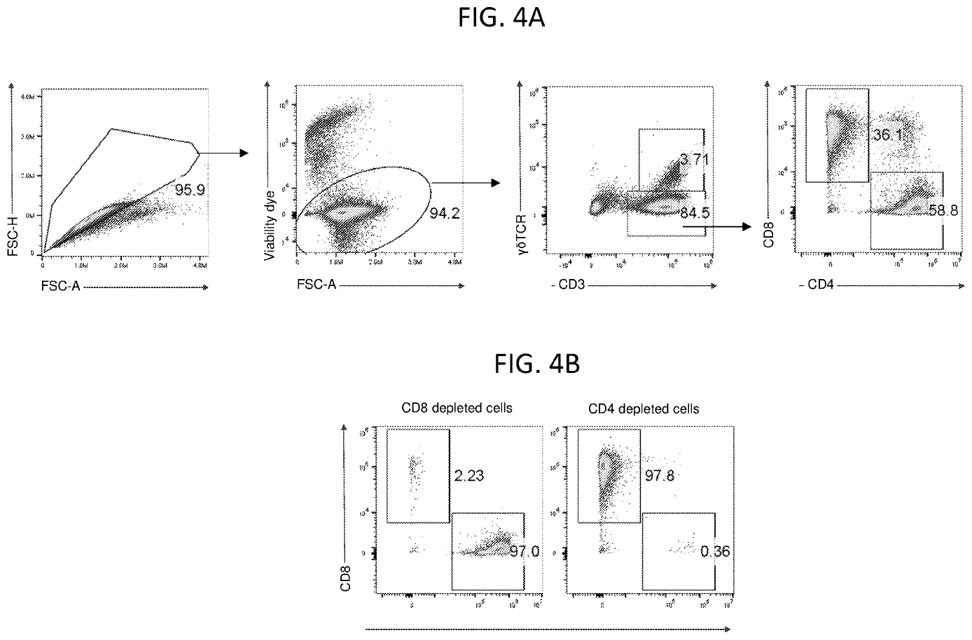

To phenotype this epitope, cells were restimulated with peptides 103-105 and intracellular cytokine flow cytometry was used to distinguish between CD4 and CD8 activation, as shown in FIG. 4A, and it was found that peptides 103-105 stimulated IFN-γ and TNF-α secretion in CD4 but not in CD8 T cells (FIG. 1C). Furthermore, the increase in IFN-γ secretion on stimulation by peptides 103-105 was no longer observed in CD4 depleted cells, whereas peptides 103-105 activated CD8 depleted cells in the ELISpot assay (FIG. 1D and FIG. 4B). These findings show that the immunodominant epitope 307-327 (peptides 103-105) is recognized by CD4 T cells.

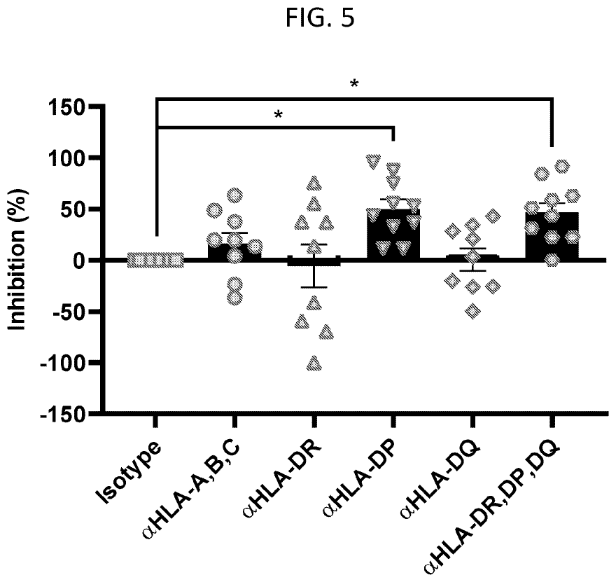

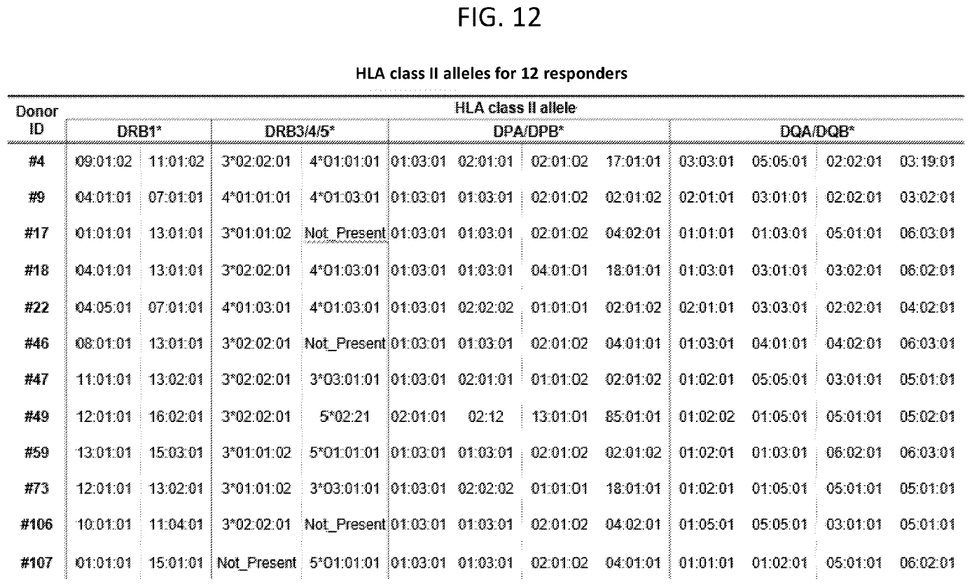

Additionally, anti-HLA-DP antibodies inhibited IFN-γ secretion while HLA-DR and HLA-DQ targeting antibodies did not (FIG. 5). The 12 donors who responded to epitope 307-327 (peptides 103-105) had a variety of HLA-DP alleles, indicating that this epitope is promiscuous (FIG. 12). Using Immune Epitope Database (IEDB) binding algorithms, the binding of epitope 307-327 (peptides 103-105) to 27 MHC class II alleles that were shown to provide maximum coverage of all potential binding cores were predicted (Martini et al., Immunogenetics 72:57-76, 2020; Greenbaum et al., Immunogenetics 63:325-335, 2011). The percentile ranks of 3 HLA-DR alleles, 1 HLA-DQ allele, and all 6 HLA-DP alleles submitted to the IEDB were below 10, which is indicative of a promiscuous binder (FIG. 13). This in silico data agreed with the HLA restriction data and HLA distribution of 12 donors further suggesting that the epitope 307-327 (peptides 103-105) is HLA-DP restricted and promiscuous.

Sequence alignment of the epitope 307-327 (peptides 103-105) region to 12 natural AAV serotypes revealed that this epitope is conserved (conservation rate: 86%) except for amino acids R312, L313, N314, F315, and L317 (FIG. 1E). Non-conserved amino acids were found in the AAV5 serotype (R299, S300, R312, V313, and I315), which is the most distantly related to AAV9 in the AAV phylogeny tree. To test whether this region in AAV5 induces immune responses, PBMCs (3 donors were chosen at random from 12 donors) were expanded with AAV5 empty capsid and restimulated with AAV5 peptides that were aligned to epitope 307-327 (peptides 103-105) in AAV9. The AAV5 peptide did not stimulate cells to produce IFN-γ, indicating that epitope 307-327 does not exist in AAV5 (FIGS. 6A-6C).

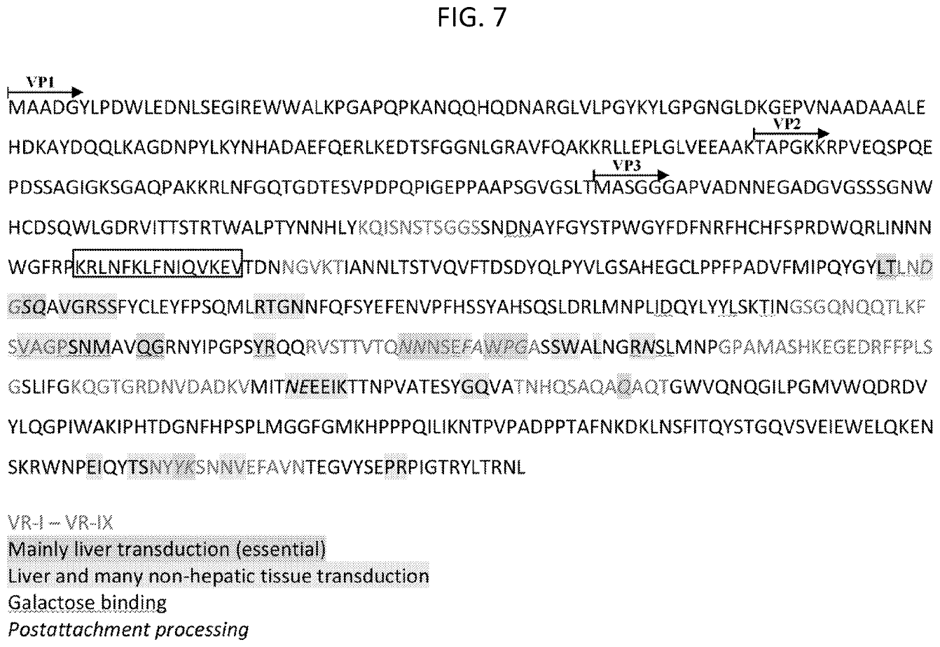

Using the IEDB, the binding affinity of AAV5 sequence to candidate amino acids responsible for the immune silencing of AAV9 immunodominant epitope was predicted. The number of HLA alleles with the modified sequences AAV9-F315V/L317I (AAV9-VI) and AAV5 (FIG. 1F and FIG. 13) having percentile ranks less than 10 was reduced to 4 and 5, respectively. Based on the predicted data, two variants of AAV9 (AAV9-VI and AAV9-RSRVI) were generated, using site-directed mutagenesis. In the AAV9-VI variant, two residues were replaced with corresponding AAV5 amino acids (F315V and L317I), and AAV9-RSRVI was designed by replacing five residues with AAV5 sequences (K311R, R312S, N314R, F315V, and L317I). These mutations have a low risk of affecting transduction efficacy or cell interaction because they are not located on the surface of the AAV9 capsid or on known functionally critical amino acids (FIG. 1G and FIG. 7).

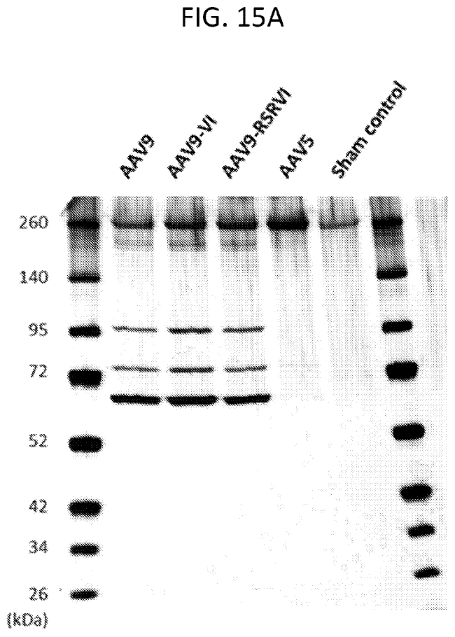

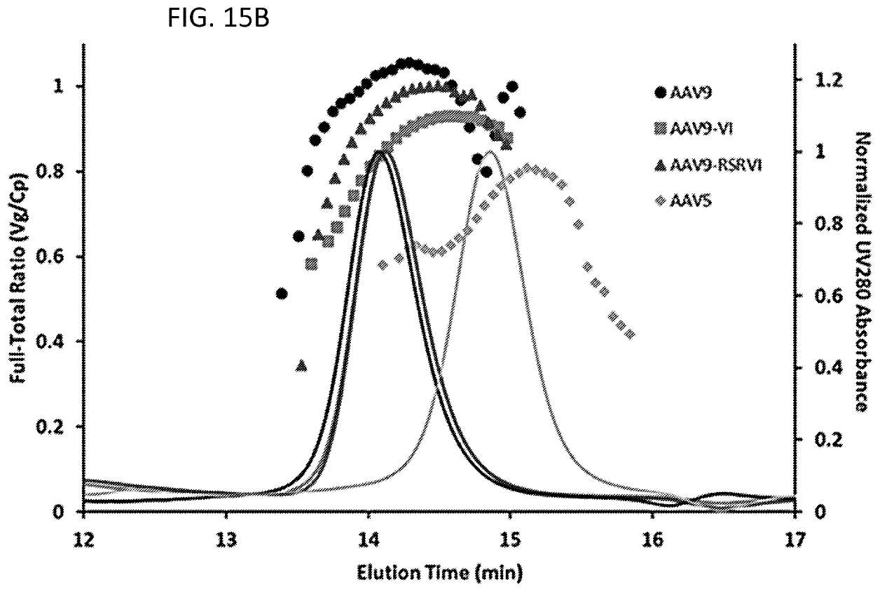

When compared to unmodified AAV5 and AAV9 capsids containing the same transgene cassette (GFP or NanoLuc luciferase), the amino acid mutations in the two AAV9 variants had no effect on the production yield, size of particle, particle thermal stability, percentage of empty capsid when manufactured by triple transfection of HEK293 cells, nor any change in the ratio between viral genome concentration and total viral particles (Table 3, Table 4, and FIGS. 15A-15B).

TABLE 3
Titer of chimeric AAV9 variants

| | | | | Capsid/viral |
|---|---|---|---|---|
| | | Quantification method | | genome ratio |
| | Transgene | qPCR (vg/mL) | ELISA (vp/mL) | (mean) |
| AAV9 | GFP* | 0.4-6.5 × 10¹¹ | 0.5-3.3 × 10¹² | 9.2 ± 3.6 |
| AAV9-VI | | 0.9-6.5 × 10¹¹ | 0.9-2.1 × 10¹² | 6.3 ± 4.4 |
| AAV9- | | 0.7-2.0 × 10¹¹ | 0.6-2.1 × 10¹² | 4.4 ± 0.1 |
| RSRVI | | | | |
| AAV5 | | 0.4-1.4 × 10¹¹ | 0.3-0.5 × 10¹² | 4.5 ± 3.5 |
| AAV9 | NanoLuc | 2.2 × 10¹² | 2.1 × 10¹² | 1.0 |
| AAV9-VI | | 2.4 × 10¹² | 3.0 × 10¹² | 1.2 |
| AAV9- | | 2.3 × 10¹² | 2.2 × 10¹² | 0.9 |
| RSRVI | | | | |
| AAV5 | | 3.2 × 10¹² | 0.6 × 10¹² | 0.2 |

*Data from 3 batches

TABLE 4
Characteristics of chimeric AAV9 variants

| | Full/Total | DLS Temp Onset | Rₕ (SEC-MALS) |
|---|---|---|---|
| | (ratio) | (°C)* | (nm) |
| AAV9 | 0.99 ± 0.03 | 74.8 ± 0.4 | 15.7 ± 1.2 |
| AAV9-VI | 0.85 ± 0.02 | 73.4 ± 0.02 | 15.7 ± 0.2 |
| AAV9-RSRVI | 0.96 ± 0.03 | 73.8 ± 0.2 | 16.1 ± 0.7 |
| AAV5 | 0.74 ± 0.07 | 78.4 ± 1.1 | 16.1 ± 1.0 |

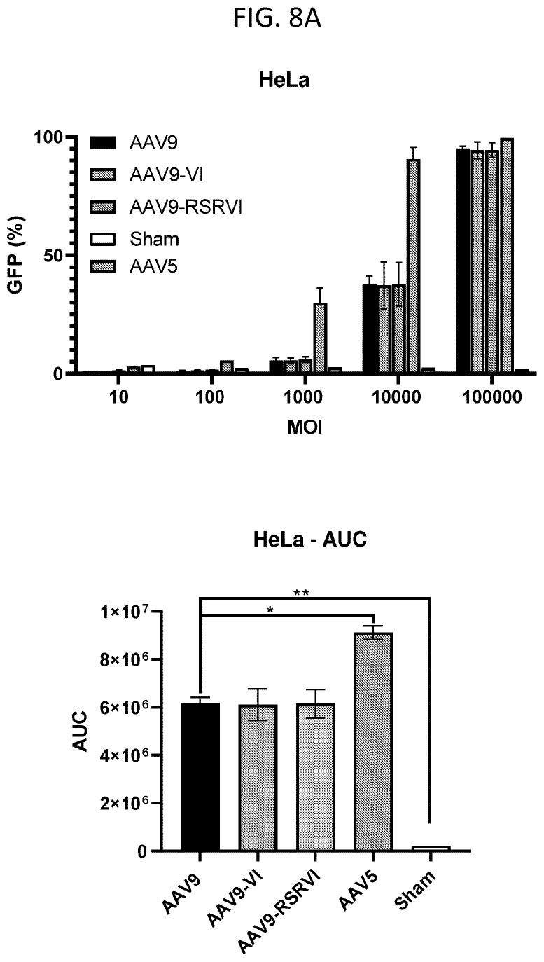

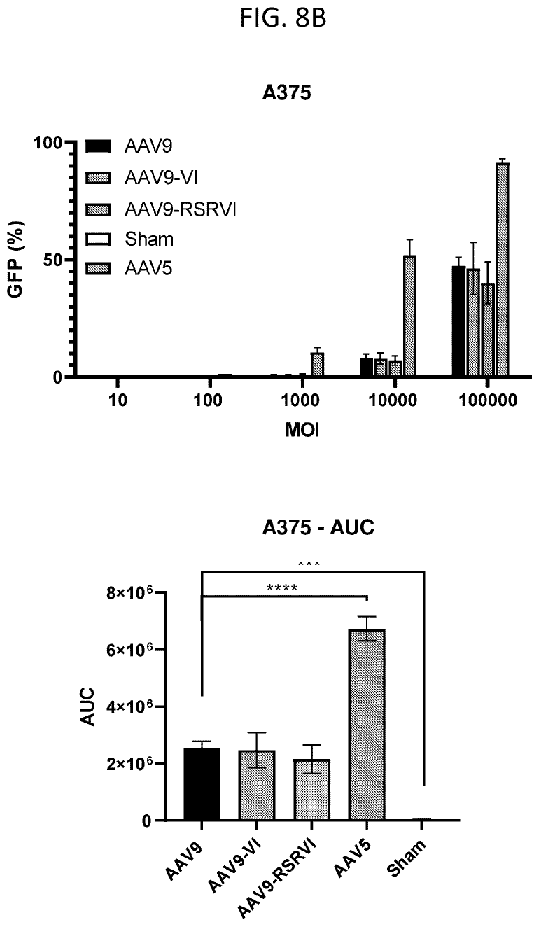

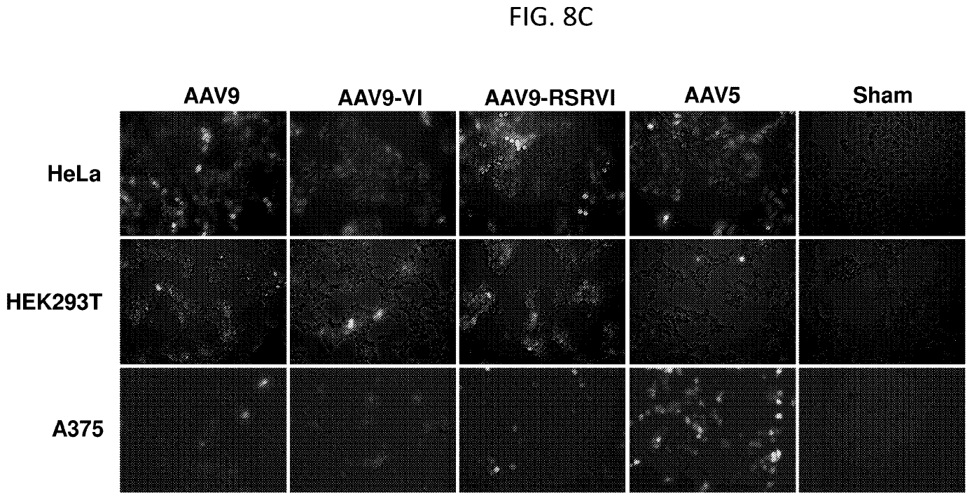

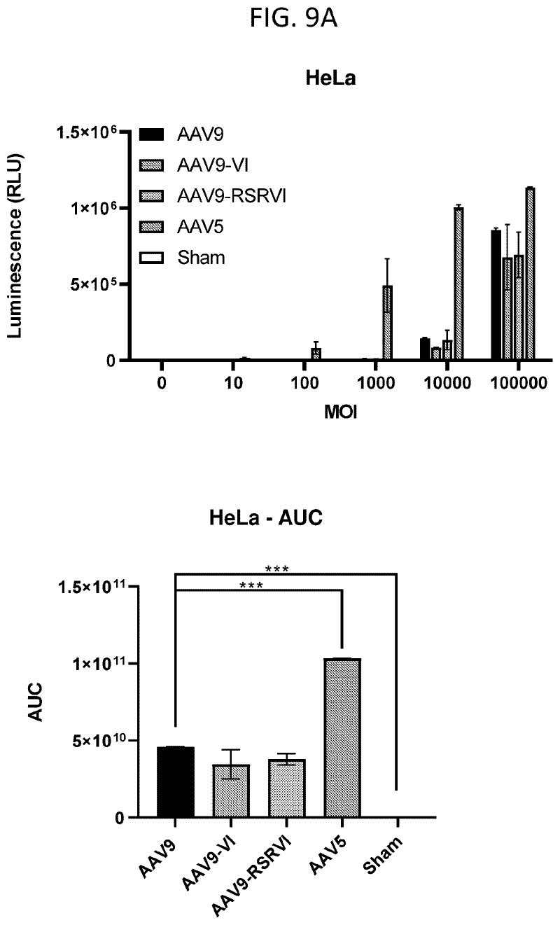

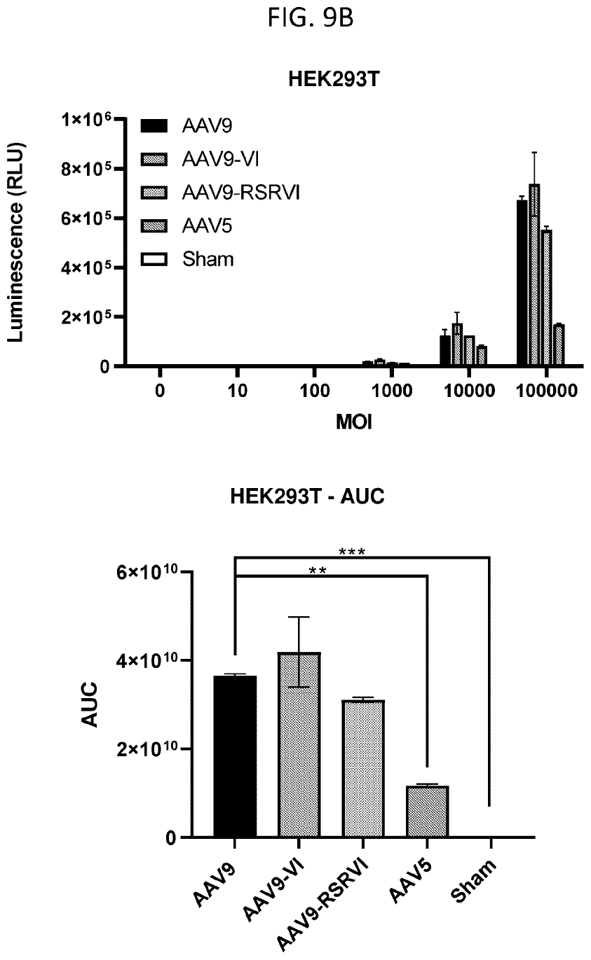

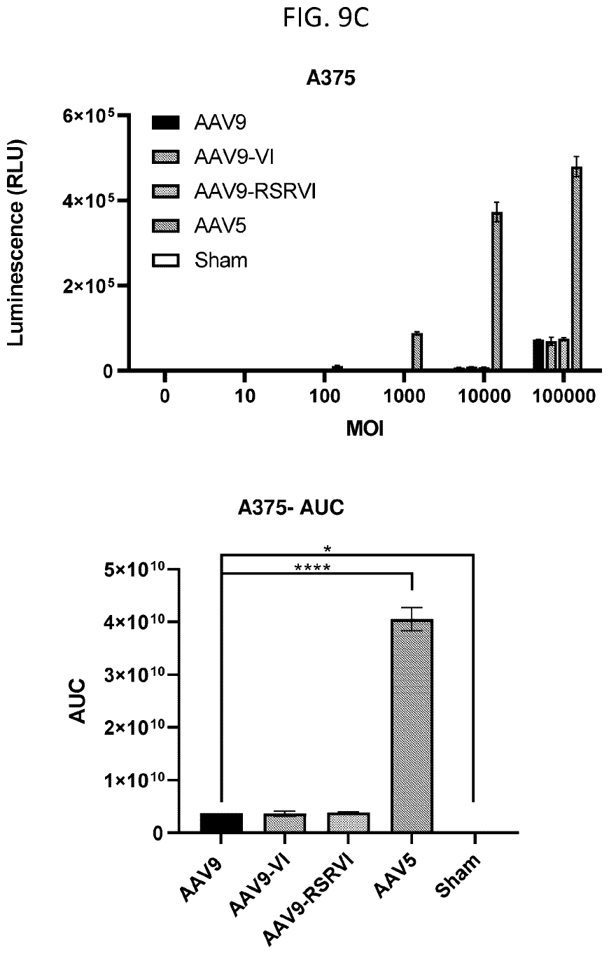

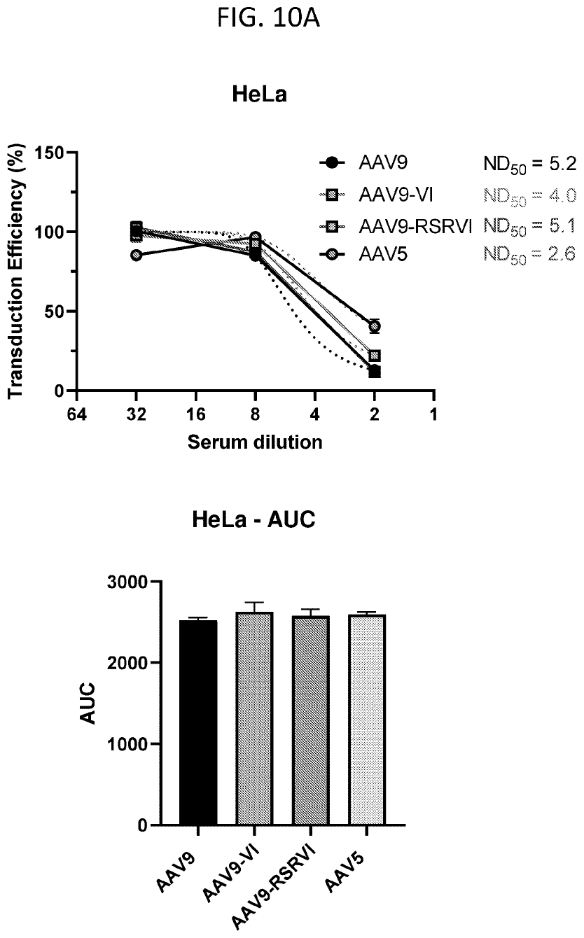

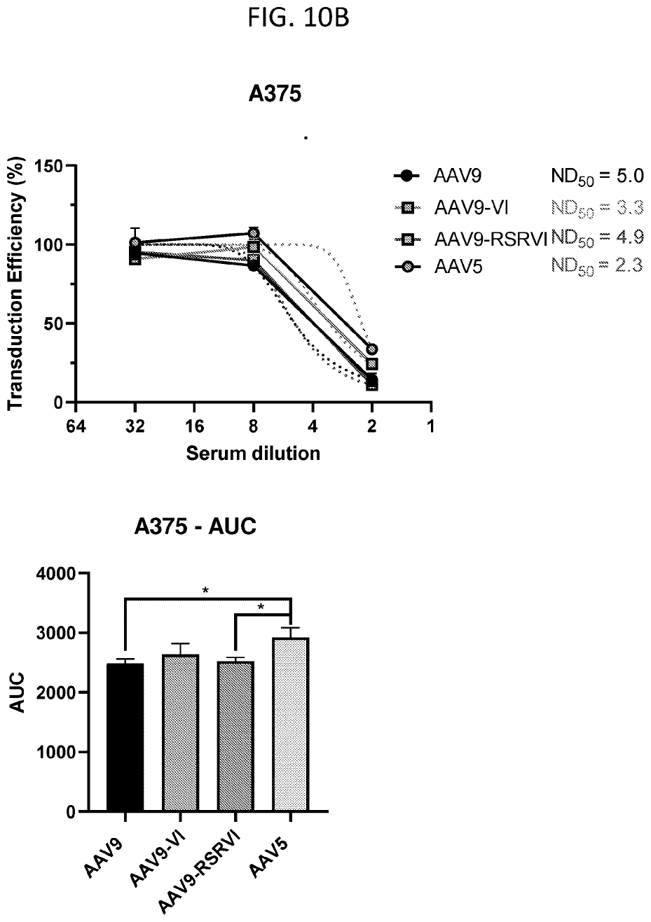

Next, the transduction profiles of the two chimeric AAV vectors in HEK293T were tested with different MOIs. The transduction efficiency of each chimeric AAV vector was analyzed and compared to the wild type (WT) AAV9-GFP and AAV5-GFP vectors using flow cytometry and fluorescence microscopy. AAV9, AAV9-VI and AAV9-RSRVI transduced cells with similar efficiency and significantly better than the AAV5 vector with 80% higher AUC (FIGS. 2A, 2B and 8C) indicating that the rational design in AAV9-VI and AAV9-RSRVI did not compromise the ability of the vector to transduce cells. Similar results were observed in other cell lines (HeLa and A375) or with a different transgene (Hall et al., ACS Chem Biol 7:1848-1857, 2012) (NanoLuc) (FIGS. 8A-8B and 9A-9C). It was then investigated whether AAV9 mutations affected the vector neutralization by anti-AAV antibodies. Before adding to the cultured cells, the vectors containing the NanoLuc transgene were incubated with a series of dilutions of pooled human serum and 50% neutralization dose (ND₅₀) was calculated. The ND₅₀ among the AAV9 and mutant vectors were not different in any of three cell lines, indicating that the mutations had no effect on vector neutralization (FIGS. 2C-2D and 10A-10B). To evaluate the transduction efficiency of the mutated vectors in vivo, vectors with the NanoLuc transgene or a sham control were injected intravenously into Balb/c mice. The levels of Luciferase expression in the mice injected with mutated vector, AAV9-VI and AAV9-RSRVI, were comparable to the AAV9 injected mice, indicating that mutations had no effect on transduction efficiency in vivo (FIG. 2E). The transgene expression in all groups remained stable after 29 days (FIG. 2F).

Because AAV tropism is a critical and sensitive parameter in capsid engineering (Adachi et al., Nat Commun 5:3075, 2014), the biodistribution of mutated vectors in the mice was also analyzed. AAV9 showed strong expression in the liver, heart, muscle, and thymus, which is consistent with previously published data (Zincarelli et al., Mol Ther 16:1073-1080, 2008), whereas expression with AAV5 was weak and mostly restricted to the liver and lung (FIG. 2G). Additional analysis for viral genome biodistribution using droplet digital PCR (ddPCR) showed the strong infection of AAV ITR in the liver, spleen, kidney, and thymus of AAV9 group (FIG. 2I). Unlike the transgene expression data, AAV9-RSRVI was transduced less in the liver and spleen, while AAV9-VI did not show any difference in the genome expressions in the tissues (FIG. 2I). Even though AAV9-RSRVI showed reduced transduction efficiency in the liver and spleen, AAV9-VI still had similar biodistribution of transgene and vector genome to that of the parental AAV vector (AAV9), indicating that the rational design had no effect on vector tropism (FIGS. 2G-2I).

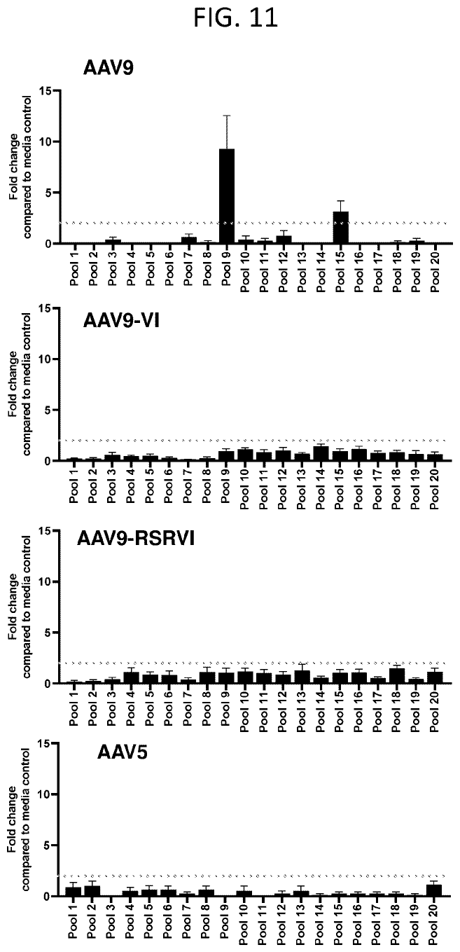

Finally, it was investigated whether the rational design eliminated the immunodominant epitope 307-327 (peptides 103-105). Restimulation of PBMCs with mutated peptides after expansion with mutated vectors did not activate the cells to produce IFN-γ, in contrast to the AAV9 expanded group, suggesting that the rational design successfully silenced the immunodominant epitope in the AAV9 capsid (FIGS. 2K-2L). One potential concern when removing an immunodominant epitope is the emergence of subdominant epitopes that were not identified when the immunodominant epitope was present (Liu et al., J Immunol 151:1852-1858, 1993). To investigate this concern, the PBMCs were stimulated with the mutated vectors and then were restimulated with the peptide library spanning all AAV9 sequences used for identification of epitope in FIG. 1A or peptides that correspond to the mutations in AAV9 VP1. FIG. 11 shows that none of the peptide pools activated the PBMCs when cells were expanded with mutated chimeric vectors, indicating that immune silencing resulted in complete elimination of the epitope without the generation of subdominant epitopes.

A further study investigated engineering of the second most prevalent epitope in pool 18 using the chimeric design strategy. Deconvolution of the individual peptides in pool 18 showed two distinct and strong epitopes in peptides 215-216 and in peptides 205-206 (FIG. 14A and Table 2). However, the equivalent peptides in AAV5 (AAV5_211-212) that were aligned to the second epitope (peptides 215-216) stimulated the cells to produce cytokines as strongly as the AAV9 peptides (AAV9_215-216) (FIG. 14B). In silico prediction also showed that the binding affinity of the AAV5 sequence that was aligned to the second epitope in the AAV9 is similar to that of the AAV9 sequence (FIG. 14C).

Collectively, these studies identified the T cell epitopes in the viral capsid protein of AAV9, including a promiscuous immunodominant epitope that can be eliminated through rational design chimerism without compromising its function and potency. Such designs can result in safer and more efficacious gene delivery vectors by reducing T cell mediated toxicities and by preventing T cell mediated death of transduced cells, potentially resulting in longer persistence of transgene expression.

It will be apparent that the precise details of the methods or compositions described may be varied or modified without departing from the spirit of the described aspects of the disclosure. We claim all such modifications and variations that fall within the scope and spirit of the claims below.

1. An isolated nucleic acid molecule encoding a modified adeno-associated virus (AAV) VP1 protein having an amino acid sequence at least 75% identical to SEQ ID NO: 1, wherein the modified VP1 protein comprises:
a valine at position 315 and an isoleucine at position 317 of SEQ ID NO: 1;
an arginine at position 311, a serine at position 312, an arginine at position 314, a valine at position 315, and an isoleucine at position 317 of SEQ ID NO: 1;
an arginine at position 314 of SEQ ID NO: 1;
an arginine at position 314 and a valine at position 315 of SEQ ID NO: 1;
an arginine at position 314 and an isoleucine at position 317 of SEQ ID NO: 1; or
an arginine at position 314, a valine at position 315 and an isoleucine at position 317 of SEQ ID NO: 1.

2. The isolated nucleic acid molecule of claim 1, wherein the AAV is AAV serotype 1 (AAV1), AAV2, AAV6, AAV7, AAV8, AAV9, AAV10 or AAV13.

3. The isolated nucleic acid molecule of claim 1, wherein:
the modified VP1 protein comprises a valine at position 315 and an isoleucine at position 317 of SEQ ID NO: 1; or
the modified VP1 protein comprises an arginine at position 311, a serine at position 312, an arginine at position 314, a valine at position 315, and an isoleucine at position 317 of SEQ ID NO: 1.

4. (canceled)

5. The isolated nucleic acid molecule of claim 1, wherein the AAV is AAV9.

6. The isolated nucleic acid molecule of claim 1, wherein the amino acid sequence of the modified VP1 protein is at least 80% identical to SEQ ID NO: 1.

7. (canceled)

8. The isolated nucleic acid molecule of claim 1, wherein the amino acid sequence of the modified VP1 protein comprises or consists of SEQ ID NO: 35 (AAV9-VI) or SEQ ID NO: 36 (AAV9-RSRVI).

9. The isolated nucleic acid molecule of claim 1, comprising:
SEQ ID NO: 37, or a degenerate variant thereof; or
SEQ ID NO: 38, or a degenerate variant thereof.

10. The isolated nucleic acid molecule of claim 1, wherein the nucleic acid sequence is codon-optimized for expression in mammalian cells.

11. A vector comprising the isolated nucleic acid molecule of claim 1.

12. (canceled)

13. The vector of claim 11, wherein the vector is an AAV vector.

14. The vector of claim 13, wherein the AAV vector is an AAV1, AAV2, AAV3, AAV6, AAV7, AAV8, AAV9, AAV10 or AAV13 vector.

15. (canceled)

16. The vector of claim 11, further comprising a heterologous open reading frame (ORF).

17. (canceled)

18. An isolated host cell comprising the isolated nucleic acid molecule of claim 1.

19. A recombinant AAV vector particle comprising the nucleic acid molecule of claim 1.

20. A recombinant adeno-associated virus (AAV) vector particle, comprising a modified VP1 protein having an amino acid sequence at least 75% identical to SEQ ID NO: 1, wherein the modified VP1 protein comprises:
a valine at position 315 and an isoleucine at position 317 of SEQ ID NO: 1;
an arginine at position 311, a serine at position 312, an arginine at position 314, a valine at position 315, and an isoleucine at position 317 of SEQ ID NO: 1;
an arginine at position 314 of SEQ ID NO: 1;
an arginine at position 314 and a valine at position 315 of SEQ ID NO: 1;
an arginine at position 314 and an isoleucine at position 317 of SEQ ID NO: 1;
an arginine at position 314, a valine at position 315 and an isoleucine at position 317 of SEQ ID NO: 1.

21. The recombinant AAV vector particle of claim 20, wherein the AAV is AAV1, AAV2, AAV6, AAV7, AAV8, AAV9, AAV10 or AAV13.

22. The recombinant AAV vector particle of claim 20, wherein:
the modified VP1 protein comprises a valine at position 315 and an isoleucine at position 317 of SEQ ID NO: 1; or
the modified VP1 protein comprises an arginine at position 311, a serine at position 312, an arginine at position 314, a valine at position 315, and an isoleucine at position 317 of SEQ ID NO: 1.

23. (canceled)

24. The recombinant AAV vector particle of claim 20, wherein the AAV is AAV9.

25. The recombinant AAV vector particle of claim 20, wherein the amino acid sequence of the modified VP1 protein is at least 80% identical to SEQ ID NO: 1.

26. (canceled)

27. The recombinant AAV vector particle of claim 20, wherein the amino acid sequence of the modified VP1 protein comprises or consists of SEQ ID NO: 35 or SEQ ID NO: 36.

28. The recombinant AAV vector particle of claim 20, further comprising an AAV genome.

29. The recombinant AAV vector particle of claim 28, wherein the AAV genome comprises a heterologous ORF.

30. The recombinant AAV vector particle of claim 29, wherein the heterologous ORF is a therapeutic gene.

31. A method of administering a therapeutic gene to a subject, comprising administering to the subject the recombinant AAV vector particle of claim 30.

32. A composition comprising the recombinant AAV vector particle of claim 20 and a pharmaceutically acceptable carrier.

33. An isolated peptide no more than 40 amino acids in length and comprising the amino acid sequence of SEQ ID NO: 3, SEQ ID NO: 4, SEQ ID NO: 5 or SEQ ID NO: 6.

34. (canceled)

35. The isolated peptide of claim 33, wherein the amino acid sequence of the peptide consists of SEQ ID NO: 3, SEQ ID NO: 4, SEQ ID NO: 5 or SEQ ID NO: 6.

36. A kit comprising:
the isolated peptide of claim 33; and
a solid support, buffer, and/or instructional material.

37. A method of inducing immune tolerance against AAV in a subject, comprising administering to the subject the isolated peptide of claim 33.

38. (canceled)

39. The method of claim 37, wherein the peptide is encapsulated in a nanoparticle or a microparticle.
